(19) United States
(12) Patent Application Publication
(10) Pub. No.: US 2026/0226481 A1
(43) Pub. Date: Aug. 6, 2026

(54) METHODS OF MAKING AND ISOLATING CIRCULAR RNAS AND CIRCULAR RNA COMPOSITIONS

(71) Applicant: THE METHODIST HOSTAL, Houston, TX (US)

(72) Inventors: Daniel Louis KISS, Pearland, TX (US); Nada BEJAR, Pearland, TX (US)

(21) Appl. No.: 19/151,031

(22) PCT Filed: Jan. 26, 2024
(86) PCT No.: PCT/US2024/013137

§ 371 Date: Jul. 25, 2025

Related U.S. Application Data
(60) Provisional application No. 63/481,944, filed on Jan. 27, 2023.

(51) Int. Cl. C12N 15/63; A61K 31/7105; C07K 14/005; C07K 16/00; C12N 9/16; C12N 15/10

(52) U.S. Cl. CPC C12N 15/63; A61K 31/7105; C07K 14/005; C07K 16/00; C12N 9/16; C12N 15/101; C12Y 301/21; C07K 2317/31; C12N 2310/16; C12N 2310/532; C12N 2830/50; C12N 2840/203

(57) ABSTRACT

Provided herein is a method of making circular RNA, a method of isolating circular RNA and compositions comprising circular RNA.

### CROSS-REFERENCE TO RELATED APPLICATIONS

This PCT application claims priority to, and the benefit of, U.S. Provisional Patent Application No. 63/481,944, filed Jan. 27, 2023, which is incorporated by reference herein in its entirety.

### REFERENCE TO SEQUENCE LISTING

The sequence listing submitted on Jan. 26, 2024, as an .XML entitled “10063-079W01_ST26.xml” created on Jan. 25, 2024, and having a file size of 100,751 bytes is hereby incorporated by reference pursuant to 37 C.F.R. § 1.52(e)(5).

### BACKGROUND OF THE INVENTION

Circular RNA (circRNA) is a promising platform in the RNA-based therapies field. Just like linear RNA, circRNA can be translated into therapeutic proteins or vaccines. However, because circRNAs lack ends, they are less susceptible to degradation by most RNAse enzymes. Therefore, they are more stable than linear RNAs and sustain a longer duration of and enable higher production of proteins per unit RNA. Increasing the duration of protein expression is particularly important for diseases requiring long-lasting therapeutic effect as well as promoting RNA vaccine's efficacy. Numerous other advantages of circRNA include (a) unlike linear RNA, they do not require the use of expensive modified nucleosides (b) they evade cellular detection of foreign RNA known to induce an immune response (c) they are known to be more thermostable than linear RNA, likely making their storage more cost-effective.

Previously, circRNAs were created using a permuted exon-intron method referred to as PIE. The PIE method involves chemical splicing with harsh conditions. These conditions lead to two major limitations: (a) an inefficient splicing, and (b) “breaks” in circRNAs leading to the accumulation of linear ‘cut-open’ circles contaminant byproducts. Accordingly, what are needed are improved methods of creating circRNAs.

### SUMMARY OF THE INVENTION

Included herein is a method of making circular RNA comprising: transcribing a vector to form a precursor RNA, wherein the vector comprises operably linked elements ordered as follows and excludes a complementarity sequence: a 3′ Group I intron or 3′ Group II intron sequence containing a 3′ splice site dinucleotide, a non-coding sequence, and a corresponding 5′ Group I intron or 5′ Group II intron sequence containing a 5′ splice site dinucleotide; and contacting the precursor RNA with a paired Group I or Group II maturase polypeptide to allow formation of the circular RNA. In some embodiments, the vector further comprises an internal ribosome entry site (IRES) and a protein coding sequence and a second non-coding sequence.

Also included herein is a method of making circular RNA comprising: transcribing a vector to form a precursor RNA, wherein the vector comprises operably linked elements ordered as follows: a 5′ complementarity sequence; a 3′ Group I intron or 3′ Group II intron sequence containing a 3′ splice site dinucleotide, a non-coding sequence, a corresponding 5′ Group I intron or 5′ Group II intron sequence containing a 5′ splice site dinucleotide, and a 3′ complementarity sequence; and contacting the precursor RNA with a paired Group I or Group II maturase polypeptide to allow formation of the circular RNA. In some embodiments, the vector further comprises an internal ribosome entry site (IRES) and a protein coding sequence and a second non-coding sequence, in that order.

In certain aspects, the maturase polypeptide and the precursor RNA are contacted under conditions comprising a temperature of between 200 C and 45° C. and a magnesium concentration of between 100 micromolar and 25 millimolar.

In some embodiments, the vector further comprises a second IRES, a second protein coding sequence, and a third non-coding sequence between the second non-coding sequence and the 5′ Group II intron sequence containing a 5′ splice site dinucleotide.

In some embodiments, the vector further comprises a third IRES, a third protein coding sequence, and a fourth non-coding sequence between the third non-coding sequence and the 5′ Group II intron sequence containing a 5′ splice site dinucleotide.

In certain aspects, the Group I intron is an Aspergillus nidulans COB1 intron. The Aspergillus nidulans I-AniI maturase can be paired with an Aspergillus nidulans COB1 intron. In some embodiments, the Aspergillus nidulans I-AniI maturase has a reduced DNA endonuclease activity and/or increased splicing activity. The Aspergillus nidulans I-AniI maturase can also be N-terminally truncated.

In some embodiments, the Group II intron is selected from Oryza sativa tmK gene, tRNAs (V-UAC, I-GAU, A-UGC, and K-UUU), ribosomal proteins (rp12 and rps12), and chloroplast ATPase (atpF). These Group II introns can be paired with Oryza sativa maturase K.

In some embodiments, the Group II intron is a Lactococcus lactis LtrA intron. The Lactococcus lactis LtrA intron can be paired with a Lactococcus lactis LtrA maturase.

In some embodiments, the Group II intron is a Lactococcus lactis LtrB intron. The Lactococcus lactis LtrB intron can be paired with a Lactococcus lactis LtrB maturase.

In some embodiments, the maturase is cyt-19 from Neurospora crassa, and the paired intron is selected from aI5γ and bI1 group II intron. In some embodiments, the maturase is cyt-19 from Saccharonyces cerevisiae and the paired intron is IIA intron aI2. In some embodiments, the maturase is Mss116p from Saccharomyces cerevisiae, and the paired intron is IIA intron aI2. In some embodiments, the maturase is MatR from the Brassicaceae plant family and the paired intron is nad1 i4. In some embodiments, the intron and paired maturase are as described in Table 2.

In certain aspects one or more of the non-coding sequences are between approximately 10 and 50 nucleotides or between approximately 20 and 30 nucleotides. In certain aspects, one or more of the non-coding sequences comprise a poly(A) sequence. In other aspects, one or more of the non-coding sequences comprise only A and C nucleotides.

The IRES element of the vector can have a sequence selected from an aptamer to eIF4G, a Homo sapiens cDNA FLJ43058, an Acute bee paralysis virus IRES, an Aphid lethal paralysis virus IRES, an Avian encephalomyelitis virus IRES, a Bovine viral diarrhea virus 1 IRES, a Canine Scamper IRES, a Classical swine fever virus IRES, a Cosavirus, a Coxsackievirus A (CVB1/2) IRES, a Coxsackievirus B3 (CVB3) IRES, a Cricket paralysis virus IRES, a Crucifer tobamo virus IRES, a Drosophila antennapedia IRES, a Diresapivirus B1 IRES, a Drosophila C Virus IRES, a Drosophila hairless IRES, a Drosophila reaper IRES, a Drosophila Ubx IRES, a Ectropis obliqua picoma-like virus IRES, a Encephalomyocarditis virus (EMCV) IRES, an Equine rhinitis virus IRES, a foot and mouth disease virus IRES, a Guanxi changeable lizard picomavirus 2 IRES, a Hepatitis A virus IRES, a Hepatitis C virus IRES, a Hepatitis GB virus IRES, a Hibiscus chlorotic ringspot virus IRES, a Himetobi P virus IRES, a Homalodisca coagulata virus-1 IRES, a Human AML1/RUNX1 gene IRES, a Human AQP4 gene IRES, a Human AT1R gene IRES, a Human BAG-1 gene IRES, a Human BCL2 gene IRES, a Human BiP gene IRES, a Human c-IAP1 gene IRES, a Human c-myc gene IRES, a Human c-src gene IRES, a Human eIF4G gene IRES, a Human enterovirus 71 IRES, a Human FGF-1 gene IRES, a Human FGF2 gene IRES, a Human Immunodeficiency Virus type 1 IRES, a Human LEF1 gene IRES, a Human n.myc gene IRES, a Human p27kip1 gene IRES, a Human p53 gene IRES, a Human papillomavirus IRES, a Human PDGF2/c-sis gene IRES, a Human Pim-1 gene IRES, a Human rhinovirus 2 IRES, a Human SFTPA1 gene IRES, a Human UNR gene IRES, a Human VEGF-A gene IRES, a Human XIAP gene IRES, a Human ELG1 IRES, a Human Caspase8-associated protein 2 (CASP8AP2), a Kashmir bee virus IRES, a Mouse eukaryotic translation initiation factor 1A domain containing 14 (Eiflad14) gene IRES, a Mouse Gtx gene IRES, a Mouse HIF1 alpha gene IRES, a Mouse NDST4L gene IRES, a Mouse Rbm3 gene IRES, a Mouse UtrA gene IRES, a Mouse Line1-ORF1 IRES., a Parechovirus IRES, a Pink-eared duck picomavirus IRES, a Plautia stali intestine virus IRES, a poliovirus 1 IRES, a Reticuloendotheliosis virus IRES, a Rhopalosiphum padi virus IRES, a Rous sarcoma virus IRES, a S. cerevisiae TFIID gene IRES, a S. cerevisiae YAP1 gene IRES, a Salivirus IRES, a Simian picomavirus IRES, a simian Virus 40 IRES, a Solenopsis invicta virus 1 IRES, a Taura syndrome virus IRES, a Theiler's encephalomyelitis virus IRES, and a Triatoma virus IRES, an Aplysia californica ELH IRES, a synthetic IRES, or a novel synthetic IRES. In some embodiments, the synthetic IRES is generated by creating a chimera added to a fragment of a known IRES. In some embodiments, the synthetic IRES is PPT19. In some embodiments, the synthetic IRES is KITI1.

In some embodiments, the protein coding sequence encodes a viral protein, a eukarvotic protein, or a prokaryotic protein. The eukaryotic protein can be a human protein. In some embodiments, the protein coding sequence encodes an antibody, such as a bispecific or monoclonal antibody. In some embodiments, the protein coding sequence encodes a viral antigen or a bacterial antigen. In other embodiments, the protein coding sequence encodes a fungal antigen or a protist antigen.

Also included herein are circular RNAs made according to the methods described herein. In some embodiments, the circular RNA comprises between 300 and 12000 nucleotides.

Further included herein are pharmaceutical compositions comprising the circular RNAs described herein and a pharmaceutically acceptable nanocarrier selected from the group consisting of a lipid nanoparticle, a lipid, a lipid polymer, a lipo-polymeric hybrid, an exosome and a leukosome.

Still further included herein are methods of isolating the circular RNAs described herein. In some embodiments, the methods comprise obtaining a mixture of linear RNA and circular RNA created by a method described herein, contacting the mixture with one or more fixed 5′ binding proteins and one or more fixed 3′ binding proteins; and isolating the circular RNA. In some embodiments, the 5′ binding protein is a Saccharomyces cerevisiae DXO 5′ binding protein, or a homolog thereof. In other embodiments, the method of isolating circular RNA comprises obtaining a mixture of linear RNA and circular RNA created by a method described herein and isolating the circular RNA using size exclusion chromatography. In other embodiments, polymerase A, RtcB RNA ligase or a polynucleotide specific for a circularization junction are used for isolation.

### BRIEF DESCRIPTION OF FIGURES

FIG. 1 is a schematic showing maturase assisted creation of a circular RNA.

FIG. 2 shows the expression of a reporter protein GFP (green fluorescent protein) from circular RNA is still increasing 48 hours post transfection, whereas the protein expressed from linear starts decreasing after 16 hours.

FIG. 3 shows the chemical splicing of A.n. Cob1 linear RNA (full size (middle cob) and shorter size (little intron) over time.

FIG. 4 (A-B) shows a purification of I-aniI maturase (A) and expression of I-aniI maturase from total bacterial lysate (B).

FIG. 5 shows tested conditions for I-aniI maturase expression in E. coli using SDS-page gel, arrowhead shows the overexpressed recombinant maturase.

FIG. 6 shows the SDS-page gel for the different steps for I-aniI maturase purification to obtain a pure recombinant I-aniI protein.

FIG. 7 is a diagram of the general mechanism of intron splicing in permuted intron exon (PIE) constructs for generating circRNA.

FIG. 8 shows that per RNAfold webserver prediction, the structure of the PIE (Permuted-Intron-Exon) version of A.n. Cob1 intron retains the same structure as the original.

FIG. 9 shows the design of PIE constructs (based on the A.n. Cob1 intron) A through E with their corresponding predicted structure using RNAfold web server.

FIG. 10 shows agarose gel (e-gel) showing initial chemical splicing reactions of tested construct A, C and E. A level of splicing is occurring for the 5′ intron (225 bases).

FIG. 11 shows the agarose gel (e-gel) of the chemical splicing of construct D at different temperatures, incubation times and buffers. At condition 6, construct D seems to be splicing more efficiently.

FIG. 12 shows the agarose gel (e-gel) of the chemical splicing of construct F while generating circRNA and splicing intermediates are increasing over time.

FIG. 13 is a schematic showing a method for isolating circular RNA using 5′ and 3′ RNA binding proteins.

FIG. 14 (A-B) shows successful recombinant protein expression for DXO, truncated-I-aniI (NusA)(A), and I-aniI (OEC-1 and OEC-2) (B). Bands of expected sizes (kD shown under each protein) with high protein expression were observed.

FIG. 15 shows that N-terminal truncation of I-aniI purification is optimized to preserve the protein from cleavage during the bacterial lysing step.

FIG. 16 (A-E) shows that in presence of I-aniI, Maturase-assisted circularization on designed constructs shows that most result in circRNA production in agreement with the structure prediction. Extended work was done to characterize construct B which was more efficient than the other constructs at producing circRNA. The resistance of the newly generated RNA entity to RNAse R treatment confirms its likelihood of being a circular RNA. (A) Construct B RNA obtained by in vitro transcription was incubated without or with the maturase I-aniI in a ratio close to 1:1 molarity (RNA to maturase) and incubated at 37° C. for 1 hour. Only in presence of the maturase, a novel band corresponding to the circRNA appears. (B) circRNA production using Construct B ribozyme and a dose increase of I-aniI show an improvement in production of circRNA (thicker circRNA band). When treated with RNAse R, which degrades linear RNA and not circRNA, we observed the resistance of the novel band to RNAse R, which confirms it is a circRNA entity. (C) All RNA for constructs A, B, C, D, E were spliced with 1:1 molar ratio with I-aniI for 1 hour:30 minutes at 37° C. circRNA band appears for A, B, and C. Construct E (negative control) did not produce a circRNA, and construct D is splicing, but the size of the circRNA band wouldn't be visible on the gel. Construct F, G and H splicing was performed were not performed at 1:1 molar ratio. Although there is a level of splicing, the reaction is not optimized. (D) Construct B was incubated in presence or absence of I-aniI maturase for the time indicated. The presence of the maturase clearly produced circRNA, with a better completion of the reaction after 160 minutes. (E) Incubation of circRNAs or precursor RNA from construct B with RNAse R results in the degradation of most of the precursor (−I-aniI+R) and the resistance of the circRNA to degradation (+I-aniI+R). This suggests the RNA entity produced is a circular RNA.

FIG. 17 shows the use of RT-PCR and Sanger sequencing to show that circRNA is being produced using maturase-assisted RNA circularization. The unique junction sequence joining the 5′ shortened exon and the 3′ shortened exon are only possible if the RNA is circular.

FIG. 18 (A-C) shows newly designed circRNA sequences (A) are able to form circRNAs in vitro (B). In this figure, “placeholder” refers to a “coding sequence.” (C) Prediction schematics of the structures of all the ribozymes tested. Each structure was plotted using RNAfold webserver with the standard parameters. A, B and C show conservation of the splicing arrangement made possible with the homology arm (grey), while the lack of the homology arm in E distorts the ribozyme structure. The predicted structures consist of an intron, exon 1, exon 2, homology arm, IRES, Nluc ORF and MCS.

FIG. 19 (A-C) shows a series of optimization experiments to increase the efficiency of circularization was performed. Constructs A, B and C successfully produce circRNAs. (A) Incubations of Construct A, B and C with I-AniI maturase for three hours at 37° C. (B) Incubations of Construct B with I-AniI maturase for 20 minutes and 45 minutes, at temperatures varying between RT to 65C, with buffers r3.1 (NEB: 100 mM NaCl, 50 mM Tris-HCL, 10 mM MgCl2, 100 μg/ml Recombinant Albumin, pH 7.9) or buffer TKM (TK+25 mM MgCl₂ (50 mM Tris pH 7, 1 mM DTT), all in presence of 1 mM GTP. (C) Incubations of Construct B with I-AniI maturase in TKM buffer for: 4 hours at 37° C. with twice as much maturase as Construct B (molar ratio) (third and fourth lanes); 3 hours at 37° C. with three times as much maturase as Construct B (molar ratio) (fifth and sixth lanes); 3 hours at 42° C. with twice as much maturase as Construct B (molar ratio) (seventh and eighth lanes).

FIG. 20 shows circRNA produced by the maturase (I-AniI) assisted circularization can be translated to a protein. Nanoluciferase activity is detected from circRNA encoding a nanoluciferase reporter protein.

FIG. 21 shows that Maturase K was cloned and is expressed.

FIG. 22 (A-B) shows recombinant LtrA maturase expression in E. coli as seen on a stain-free SDS-PAGE gel and by western detection with an anti-V5 antibody (A). Recombinant LtrA maturase is purified from E. coli cell lysate (B). Arrows mark the expected LtrA fusion protein band.

FIG. 23 (A-C) shows self-splicing and maturase-assisted RNA circularization profiles for P2.1 as working control and P2.2 (A). P2.1 construct is P2 with a 15-nucleotide extension on the 5′ exon (to create a long non-coding sequence) and P2.2 construct is P2.1 with a 15-nucleotide extension on the 3′ exon (to create a long non-coding sequence) to facilitate RNA circularization. RNA circularization profiles for P2.1 and P2.2 after adjusting the incubation times and assaying at a 1:4 ratio of RNA to LtrA maturase as seen on a 1.2% FlashGel™ RNA cassette (B). Double asterisks indicate the circRNA. Detection of the novel circRNA-specific sequence junction using reverse transcriptase PCR followed by Sanger sequencing demonstrates that the circRNA is an intact circle (C).

FIG. 24 (A-D) shows different sequence designs to circRNAs using LtrA maturase, wherein “D” refers to an RNA domain of a LtrB intron (A). RNA structure predictions generated by RNAfold for both the linear control RNA (Lin1) and RNA circularization constructs during initial design, validation, and optimization stages of the project (B-C). The designations D1, D2, D3, D4, D5, D6 in (A) correspond to the RNA domains numbered I, II, III, IV, V, and VI, respectively shown in schematic provided in (D).

FIG. 25 (A-B) shows RNA circularization profile for P2 and splicing profile for Lin1 after adjusting the incubation times and assaying at a 1:2 ratio of RNA to LtrA maturase (A). Maturase-assisted circRNA generation using optimized circularization sequences (P2.1 construct is P2 with an extension to facilitate RNA circularization) and splicing profiles for the Lin1 linear RNA control (B). Double asterisks indicate the circRNA.

FIG. 26 shows LtrA maturase-assisted circular RNAs can express an encoded protein. Nanoluciferase reporter activity in U2OS-K294A cells transfected with LtrA maturase-assisted circRNA (circ5. lNluc) and a reference circRNA (circNluc).

FIG. 27 (A-C) shows recombinant MatR maturase expression in E. coli as seen on a stain-free SDS-PAGE gel and by western detection with an anti-V5 antibody (A). Different conditions are evaluated to optimize MatR expression in E. coli (B). Recombinant MatR maturase is purified from E. coli cell lysates as seen on a stain-free SDS-PAGE gel (C). Arrows mark the expected MatR maturase protein.

FIG. 28 (A-B) shows Cyt-19 dead-box protein is highly expressed in bacteria (A) and was successfully purified from E. coli lysates (B).

FIG. 29 shows increasing amounts of cyt19 protein in circularization reactions with circRNA construct B (described in FIGS. 17-19) did not show a positive effect on promoting circRNA formation.

FIG. 30 shows detection of the novel circRNA-specific sequence junction using reverse transcriptase PCR followed by Sanger sequencing demonstrates that the circRNA is an intact circle. This particular circRNA design uses non-coding RNA sequences to boost the efficiency of circRNA production.

FIG. 31 (A-C) shows a schematic of the plasmid backbone for generating the internal ribosome entry site (IRES) test pool library (maximum 10⁸ unique 150 nucleotide random sequences). Each random 150-nt sequence is placed in front of the mCherry reporter protein coding sequence (A). circRNAs were generated using linearized plasmid library as a template (B). Microscope images showing that a small number of cells can express mCherry reporter protein from transfected circRNAs. Only circRNAs with a functional IRES sequence should be able to generate mCherrv protein in cells (C).

FIG. 32 shows the isolation of pure circRNA using a circRNA Capture Method. A biotinylated locked nucleic acid oligonucleotide (TT177) specific to the circularization junction of the RNA was hybridized to the circRNA and used to purify the intact circRNAs (marked by an oval) away from linear RNA contaminants (bar). A 2-hour incubation saw good recovery of circRNA with the streptavidin beads, and the circRNAs were intact.

FIG. 33 (A-B) shows isolation of pure circRNA using RNA ligase and Desthiobiotin followed by recovery using streptavidin beads (linear RNA Capture Method #1) and used to purify the intact circRNAs (marked by an oval) away from linear RNA contaminants (bars). The experiment was performed using both crude circRNA preparations (A) and purer circRNA preparations (B). Little enrichment of circRNAs was observed.

FIG. 34 shows isolation of pure circRNA using RtcB protein to bind and capture linear RNA byproducts (linear RNA Capture Method #2). The circRNAs do pass through the procedure unmodified (sample 5 in different lanes of the two gels) with some loss in amount. Results do show a reduction in the major linear RNA species (faint band in sample 1, bottom gel) but the ability to capture the linear RNA byproducts was not quantitatively evaluated.

FIG. 35 (A-B) shows isolation of pure circRNA using DXO protein to bind and capture linear RNA byproducts (linear RNA Capture Methods #3.1 and #3.2) Part One. Expression (A) and purification (B) of wild type and exonuclease mutant DXO proteins from E. coli cell lysates. Arrows mark the expected WT and D236A/E253A mutant DXO protein bands (expected 54 kDa).

FIG. 36 (A-B) shows isolation of pure circRNA using DXO protein to bind and capture linear RNA byproducts (linear RNA Capture Methods #3.1 and #3.2) Part Two. Exoribonuclease assays were performed on two different circRNAs using WT and D236A/E253A (nuclease inactive) mutant DXO. The purified WT DXO has nuclease activity as shown by the RNA cleavage products (brackets) in two different RNA substrates treated in two ways (circESAT6 IVT RNA (A) and circGFP-3XFL IVT RNA, (B). These cleavage products are not seen in the mutant DXO treatment, suggesting that the nuclease activity is abolished in the generated D236A/E253A mutant DXO.

FIG. 37 (A-B) shows isolation of pure circRNA using poly(A) polymerase to extend linear RNA byproducts with biotinylated adenosines which will be used to bind and capture the linear RNA byproducts (linear RNA Capture Method #4). The products after poly(A) extension of a mixed pool of circRNAs and linear RNA substrates (A, short arrows show linear byproducts and long color-matched arrows show polyadenylated linear RNA byproducts after the procedure). The resulting samples were assayed using a TapeStation chromatogram (B). The intensity of samples resolved in each lane of chromatogram in (B) is shown in (C-H).

### DETAILED DESCRIPTION

Provided herein are methods of making circular RNA that significantly improve upon methods in the prior art. In some embodiments, the methods are both more efficient and more effective than methods of the prior art. Also included herein are methods of isolating the circular RNAs described herein, circular RNA compositions, and pharmaceutical compositions comprising the circular RNA compositions. The methods provided herein make use of maturases, and more specifically, maturase intron pairs, which allow for the use of milder reaction conditions and improved circRNA yield.

Terms used throughout this application are to be construed with ordinary and typical meaning to those of ordinary skill in the art. However, Applicant desires that the following terms be given the particular definition as defined below.

### Definitions.

As used in the specification and claims, the singular form “a,” “an,” and “the” include plural references unless the context clearly dictates otherwise. For example, the term “a cell” includes a plurality of cells, including mixtures thereof.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 Daltons, composed of two identical light (L) chains and two identical heavy (H) chains. Each heavy chain has at one end a variable domain (VH) followed by a number of constant domains. Each light chain has a variable domain at one end (VL) and a constant domain at its other end.

The term “antibody fragment” refers to a portion of a full-length antibody, generally the target binding or variable region. Examples of antibody fragments include Fab, Fab′. F(ab′)₂ and Fv fragments. The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, FcεR1. As used herein, “functional fragment” with respect to antibodies, refers to Fv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (VII-VL dimer). It is in this configuration that the three CDRs of each variable domain interact to define a target binding site on the surface of the VH-VL dimer. Collectively, the six CDRs confer target binding specificity to the antibody. However, even a single variable domain (or half of an Fv comprising only three CDRs specific for a target) has the ability to recognize and bind the target, although at a lower affinity than the entire binding site. “Single-chain Fv” or “sFv” antibody fragments comprise the VH and VL domains of an antibody, wherein these domains are present in a single polypeptide chain. Generally, the Fv polypeptide further comprises a polypeptide linker between the VH and VL domains which enables the sFv to form the desired structure for target binding.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

A “decrease” or “reduction” can refer to any change that results in a smaller amount of a symptom, disease, composition, condition, or activity. A decrease or reduction can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Homologs” are defined herein as two polynucleotides or two polypeptides that have some identity. Homologs include allelic variants, orthologs, and paralogs having the same relevant function (e.g., ability to function as maturase). In some embodiments, homologs have about 99%. 98%. 97%. 96%. 95%. 94%. 93%, 92, 91% or 90% identity. In other embodiments, homologs have about 80% or about 85% homology. In other embodiments, homologs have about 50% identity. In some embodiments, homologs have about 50%, 60%, 70% or 80% identity.

The term “identity” shall be construed to mean the percentage of nucleotide bases or amino acid residues in the candidate sequence that are identical with the bases or residues of a corresponding sequence to which it is compared, after aligning the sequences and introducing gaps, if necessary to achieve the maximum percent identity for the entire sequence, and not considering any conservative substitutions as part of the sequence identity. Neither N- nor C-terminal extensions nor insertions shall be construed as reducing identity or homology. A polynucleotide or polynucleotide region (or a polypeptide or polypeptide region) that has a certain percentage (for example, 80%, 85%, 90%, or 95%) of “sequence identity” to another sequence means that, when aligned over their full lengths, that percentage of bases (or amino acids) are the same in comparing the two sequences. It should be understood that alignment over “their full lengths” applies not only to full length polynucleotides, but also polynucleotide regions. Such polynucleotide regions are referred to herein as “conserved regions.” This alignment and the percent sequence identity can be determined using software programs known in the art. In one embodiment, default parameters are used for alignment. In one embodiment a BLAST program is used with default parameters. In one embodiment, BLAST programs BLASTN and BLASTP are used with the following default parameters: Genetic code=standard; filter=none: strand=both; cutoff=60; expect=10; Matrix=BLOSUM62; Descriptions=50 sequences; sort by=HIGH SCORE: Databases=non-redundant, GenBank+EMBL+DDBJ+PDB+GenBank CDS translations+SwissProtein+SPupdate+PIR.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

The word “maturase” is used herein to refer to any protein that can assist or facilitate intron splicing. In some embodiments, the maturase is an RNA chaperone such as cyt-19.

“Non-coding sequence” refers herein to a polynucleotide sequence that does not encode an amino acid sequence.

A “pharmaceutical composition” is intended to include the combination of an active agent with a pharmaceutically acceptable carrier, inert or active, making the composition suitable for diagnostic or therapeutic use in vivo or ex vivo.

The terms “pharmaceutically acceptable carrier” and “pharmaceutically acceptable nanocarrier” mean a carrier or excipient that is useful in preparing a pharmaceutical composition that is generally safe and non-toxic and includes a carrier that is acceptable for veterinary and/or human pharmaceutical use. As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. As used herein, the term “carrier” encompasses any excipient, diluent, filler, salt, buffer, stabilizer, solubilizer, lipid, stabilizer, or other material well known in the art for use in pharmaceutical formulations and as described further below. Pharmaceutical compositions also can include preservatives. A “pharmaceutically acceptable carrier” or a “pharmaceutically acceptable nanocarrier” as used in the specification and claims includes both one and more than one such carrier. In some embodiments, the pharmaceutically acceptable nanocarrier is a lipid nanoparticle, a lipid, a lipid polymer, a lipo-polymeric hybrid, an exosome or a leukosome.

As used herein, the term “protein coding sequence” refers to a polynucleotide sequence that encodes a polypeptide, including a fragment of a known functional polypeptide. Similarly, the word “protein” includes entire proteins and fragments of proteins.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The word “vector” refers to a vehicle that carries a polynucleotide into a cell for the expression of the polynucleotide in the cell. Once transformed into a suitable host, the vector may replicate and function independently of the host genome, or may in some instances, integrate into the genome itself. In some embodiments, the vector is a DNA construct containing a DNA sequence which is operably linked to a suitable control sequence capable of affecting the expression of the DNA in a suitable host cell. In some embodiments, such control sequences can include a promoter to effect transcription, an optional operator sequence to control such transcription, a sequence encoding suitable mRNA ribosome binding sites, and sequences which control the termination of transcription and translation.

### DESCRIPTION

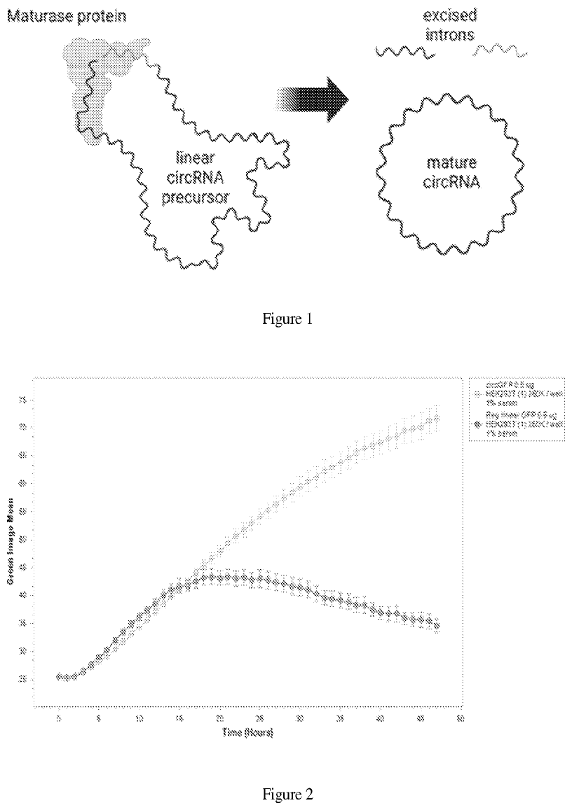

As noted above, provided herein are methods of making circular RNA that significantly improve upon methods in the prior art. Also included herein are methods of isolating the circular RNAs described herein, circular RNA compositions, and pharmaceutical compositions comprising the circular RNA compositions. It is a surprising finding that the use of maturase intron pairs, among other things, increases the splicing efficiency and reduces the formation of byproducts. FIG. 1 provides a general schematic of the maturase assisted process for creating circular RNA.

In some embodiments, the method of making circular RNA comprises the following steps:
a. transcribing a vector to form a precursor RNA, wherein the vector comprises operably linked elements ordered as follows and excludes a complementarity sequence:
   i. a 3′ Group I intron or 3′ Group II intron sequence containing a 3′ splice site dinucleotide,
   ii. a non-coding sequence, and
   iii. a corresponding 5′ Group I intron or 5′ Group II intron sequence containing a 5′ splice site dinucleotide; and
b. contacting the precursor RNA with a paired Group I or Group II maturase polypeptide to allow formation of the circular RNA.

It should be understood that for all embodiments described herein, the steps of transcribing and contacting can occur simultaneously or sequentially. In some embodiments, the contacting step begins before transcription is complete.

In some embodiments, the method of making circular RNA comprises the following steps:
a. transcribing a vector to form a precursor RNA, wherein the vector comprises operably linked elements ordered as follows and excludes a complementarity sequence:
   i. a 3′ Group I intron or 3′ Group II intron sequence containing a 3′ splice site dinucleotide,
   ii. a first non-coding sequence,
   iii. a protein coding sequence,
   iv. a second non-coding sequence, and
   v. a corresponding 5′ Group I intron or 5′ Group II intron sequence containing a 5′ splice site dinucleotide; and
b. contacting the precursor RNA with a paired Group I or Group II maturase polypeptide to allow formation of the circular RNA.

In some methods, the vector further comprises an internal ribosome entry site (IRES) and a protein coding sequence and a second non-coding sequence, in that order, between the elements of a. ii. and a.iii. Thus, included herein are methods method of making circular RNA comprising the following steps:
a. transcribing a vector to form a precursor RNA, wherein the vector comprises operably linked elements ordered as follows and excludes a homology sequence:
   i. a 3′ Group I intron sequence or 3′ Group II intron sequence containing a 3′ splice site dinucleotide,
   ii. a first non-coding sequence,
   iii. an internal ribosome entry site (IRES),
   iv. a protein coding sequence,
   v. a second non-coding sequence,
   vi. a corresponding 5′ Group I intron sequence or 5′ Group IL intron sequence containing a 5′ splice site dinucleotide; and
b. contacting the precursor RNA with a paired Group I maturase or paired Group II maturase polypeptide to allow formation of the circular RNA.

As used herein, “Group I intron” refers to a self-splicing ribozyme that can catalyze its own excision from a precursor RNA. Group I introns commonly consist of nine paired regions P1-P9 that often fold into two domains, the P4-P6 domain formed from the stacking of P5, P4, P6 and P6a helices and the P3-P9 domain formed from the P8, P3, P7 and P9 helices. “Group II intron” also refers to a self-splicing ribozyme that can catalyze its own excision from a precursor RNA. However, Group II introns differ from Group I introns in that excision can occur in the absence of GTP. Group II introns commonly have a secondary structure of six stem-loop structures D1 to D6, wherein the domains radiate from a central core that brings the 5′ and 3′ splice junctions into close proximity.

The term “3′ Group I intron sequence” refers to any DNA sequence that encodes a Group I intron RNA sequence that includes a 3′ end of a Group I intron and does not include a 5′ end of a Group I intron. The term “3′ Group II intron sequence” refers to any DNA sequence that encodes a Group II intron RNA sequence that includes a 3′ end of a Group II intron and does not include a 5′ end of a Group II intron. The term “5′ Group I intron sequence” refers to any DNA sequence that encodes a Group I intron RNA sequence that includes a 5′ end of a Group I intron and does not include a 3′ end of a Group I intron. The term “5′ Group II intron sequence” refers to any DNA sequence that encodes a Group II intron RNA sequence that includes a 5′ end of a Group II intron and does not include a 3′ end of a Group II intron. In some embodiments, the 5′ or 3′ Group I or II sequences contains deletions. One example of such deletion is in the “minimal D2 and D3” element in construct P2. In some embodiments, the 5′ or 3′ Group I or II sequences are about 100 to 750 nucleotides in length. A “corresponding 5′ Group I intron sequence or 5′ Group II intron sequence” means a 5′ Group I or Group II intron sequence from the same Group I or Group II intron, respectively, as the aforementioned 3′ Group I intron sequence or 3′ Group II intron sequence. In some embodiments, the 3′ and 5′ intron sequences are each approximately one half of a Group I or Group II intron. In some embodiments, the 3′ and 5′ intron sequences are the result of differential splitting of a Group I or Group II intron. The 3′ and 5′ splice site dinucleotides can be any dinucleotide that is the site of splicing by a Group 1 intron or a Group II intron. In some embodiments, the 5′ splice site dinucleotide is GT.

In some aspects, the 3′ Group I intron sequence comprises SEQ ID NO:27, SEQ ID NO:30, SEQ ID NO:32, or SEQ ID NO:34. Accordingly, in some embodiments, the 3′ Group I intron sequence comprises or is SEQ ID NO:27. in some embodiments, the 3′ Group I intron sequence comprises or is SEQ ID NO:30. in some embodiments, the 3′ Group I intron sequence comprises or is SEQ ID NO: 32. in some embodiments, the 3′ Group I intron sequence comprises or is SEQ ID NO:34. In some aspects, the 5′ Group I intron sequence comprises or is SEQ ID NO:28, SEQ ID NO:29, SEQ ID NO:31. SEQ ID NO:33, or SEQ ID NO:35. Accordingly, in some embodiments, the 5′ Group I intron sequence comprises or is SEQ ID NO:29. In some embodiments, the 5′ Group I intron sequence comprises or is SEQ ID NO:31. In some embodiments, the 5′ Group I intron sequence comprises or is SEQ ID NO:33. In some embodiments, the 5′ Group I intron sequence comprises or is SEQ ID NO:35.

Accordingly, in some embodiments, the method of making circular RNA comprises the following steps:
a. transcribing a vector to form a precursor RNA, wherein the vector comprises operably linked elements ordered as follows and excludes a complementarity sequence:
   i. a 3′ Group I intron sequence containing a 3′ splice site dinucleotide,
   ii. anon-coding sequence, and
   iii. a corresponding 5′ Group I intron sequence containing a 5′ splice site dinucleotide; and
b. contacting the precursor RNA with a paired Group I maturase polypeptide to allow formation of the circular RNA.

In other embodiments, the method comprises the method of making circular RNA comprises the following steps:
a. transcribing a vector to form a precursor RNA, wherein the vector comprises operably linked elements ordered as follows and excludes a complementarity sequence:
   i. a 3′ Group II intron sequence containing a 3′ splice site dinucleotide,
   ii. a non-coding sequence, and
   iii. a corresponding 5′ Group II intron sequence containing a 5′ splice site dinucleotide; and
b. contacting the precursor RNA with a paired Group II maturase polypeptide to allow formation of the circular RNA.

In some methods, the vector further comprises an internal ribosome entry site (IRES) and a protein coding sequence and a second non-coding sequence, in that order, between the elements of a. ii. and a.iii.

Accordingly, included herein are methods of making circular RNA comprising the following steps:
a. transcribing a vector to form a precursor RNA, wherein the vector comprises operably linked elements ordered as follows and excludes a complementarity sequence:
   i. a 3′ Group I intron sequence containing a 3′ splice site dinucleotide,
   ii. a first non-coding sequence,
   iii. an internal ribosome entry site (IRES),
   iv. a protein coding sequence,
   v. a second non-coding sequence,
   vi. a corresponding 5′ Group I intron sequence containing a 5′ splice site dinucleotide; and
b. contacting the precursor RNA with a paired Group I maturase polypeptide to allow formation of the circular RNA, wherein the 3′ splice site dinucleotide and the 5′ splice site dinucleotide are sites of splicing by a Group I intron.

In other embodiments, the method of making circular RNA comprises the following steps:
a. transcribing a vector to form a precursor RNA, wherein the vector comprises operably linked elements ordered as follows and excludes a complementarity sequence:
   i. a 3′ Group II intron sequence containing a 3′ splice site dinucleotide,
   ii. a first non-coding sequence,
   in. an internal ribosome entry site (IRES),
   iv. a protein coding sequence,
   v. a second non-coding sequence,
   vi. a corresponding 5′ Group II intron sequence containing a 5′ splice site dinucleotide; and
b. contacting the precursor RNA with a paired Group II maturase polypeptide to allow formation of the circular RNA, wherein the 3′ splice site dinucleotide and the 5′ splice site dinucleotide are sites of splicing by a Group II intron.

The non-coding sequences of the vector are each any DNA sequence that does not code for an amino acid. In some embodiments, the non-coding sequences have a length of between about 15 and 50 nucleotides or about 20 and 30 nucleotides. In some aspects, the non-coding sequences comprise a poly(A) sequence. In other or further embodiments, the non-coding sequences comprise only A and C nucleotides. In some embodiments, each non-coding sequence is the same. In other embodiments, each non-coding sequence is different. The “non-coding” sequences are also referred to herein as “exons.” For example, the each “exon” in FIG. 9 is or comprises a non-coding sequence. In some embodiments, a “short” non-coding sequence or exon is about 15-25 or 18-22 nucleotides in length. In other or further embodiments, the “long” non-coding sequence or exon is about 39 nucleotides in length. In some embodiments, the first non-coding sequence is or comprises SEQ ID NO:38 or SEQ ID NO:39. In some embodiments, the second non-coding sequence is or comprises SEQ ID NO:42 or SEQ ID NO:43.

The “protein coding sequence” of the vector is any DNA sequence that codes for one or more amino acids. Accordingly, it should be understood that the term “protein coding sequence” includes DNA sequences that encode fragments or portions of full proteins. In some embodiments, the protein coding sequence encodes a viral protein, a eukaryotic protein, a prokaryotic protein, a fungal protein, or a protozoan protein, which includes viral antigens, bacterial antigens, fungal antigens, and protozoan antigens. In some embodiments, the protein coding sequence encodes a human protein. In some aspects, the protein coding sequence encodes an antibody. The antibody can be any antibody, including as that term is defined herein, and in some aspects, the antibody is bispecific. In some embodiments, the protein coding sequence encodes more than one protein or protein fragment.

The present disclosure includes vectors that comprise more than one protein coding sequence. In some embodiments, the vector comprises one, two or three coding sequences. Accordingly, included herein are methods of making a circular RNA wherein the vector further comprises a second IRES, a second protein coding sequence, and a third non-coding sequence between the second non-coding sequence and the 5′ Group I or Group II intron sequence containing a 5′ splice site dinucleotide. In still further embodiments, the vector comprises a third IRES, a third protein coding sequence, and a fourth non-coding sequence between the third non-coding sequence and the 5′ Group I or Group II intron sequence containing a 5′ splice site dinucleotide.

The internal ribosome entry site (IRES) is an RNA sequence that allows for translation initiation. In some embodiments, the IRES has a sequence of an aptamer to eIF4G, a Homo sapiens cDNA FLJ43058, an Acute bee paralysis virus IRES, an Aphid lethal paralysis virus IRES, an Avian encephalomyelitis virus IRES, a Bovine viral diarrhea virus I IRES, a Canine Scamper IRES, a Classical swine fever virus IRES, a Cosavirus, a Coxsackievirus A (CVB1/2) IRES, a Coxsackievirus B3 (CVB3) IRES, a Cricket paralysis virus IRES, a Crucifer tobamo virus IRES, a Drosophila antennapedia IRES, a Diresapivirus B1 IRES, a Drosophila C Virus IRES, a Drosophila hairless IRES, a Drosophila reaper IRES, a Drosophila Ubx IRES, a Ectropis obliqua picoma-like virus IRES, a Encephalomyocarditis virus (EMCV) IRES, an Equine rhinitis virus IRES, a foot and mouth disease virus IRES, a Guanxi changeable lizard picomavirus 2 IRES, a Hepatitis A virus IRES, a Hepatitis C virus IRES, a Hepatitis GB virus IRES, a Hibiscus chlorotic ringspot virus IRES, a Himetobi P virus IRES, a Homalodisca coagulata virus-1 IRES, a Human AML1/RUNX1 gene IRES, a Human AQP4 gene IRES, a Human ATIR gene IRES, a Human BAG-1 gene IRES, a Human BCL2 gene IRES, a Human BiP gene IRES, a Human c-IAP1 gene IRES, a Human c-myc gene IRES, a Human c-src gene IRES, a Human eIF4G gene IRES, a Human enterovirus 71 IRES, a Human FGF-1 gene IRES, a Human FGF2 gene IRES, a Human Immunodeficiency Virus type 1 IRES, a Human LEF1 gene IRES, a Human n.mvc gene IRES, a Human p27kip1 gene IRES, a Human p53 gene IRES, a Human papillomavirus IRES, a Human PDGF2/c-sis gene IRES, a Human Pim-1 gene IRES, a Human rhinovirus 2 IRES, a Human SFTPA1 gene IRES, a Human UNR gene IRES, a Human VEGF-A gene IRES, a Human XIAP gene IRES, a Kashmir bee virus IRES, a Mouse eukaryotic translation initiation factor 1A domain containing 14 (Eiflad14) gene IRES, a Mouse Gtx gene IRES, a Mouse HIF1 alpha gene IRES, a Mouse NDST4L gene IRES, a Mouse Rbm3 gene IRES, a Mouse UtrA gene IRES, a Parechovirus IRES, a Pink-eared duck picornavirus IRES, a Plautia stali intestine virus IRES, a poliovirus 1 IRES, a Reticuloendotheliosis virus IRES, a Rhopalosiphum padi virus IRES, a Rous sarcoma virus IRES, a S. cerevisiae TFIID gene IRES, a S. cerevisiae YAP1 gene IRES, a Salivirus IRES, a Simian picomavirus IRES, a simian Virus 40 IRES, a Solenopsis invicta virus 1 IRES, a Taura syndrome virus IRES, a Theiler's encephalomyelitis virus IRES, or a Triatoma virus IRES. In some embodiments, the IRES is a Coxsackievirus B3 (CVB3) IRES. In certain aspects, the CVB3 IRES comprises SEQ ID NO:36. In some embodiments, the IRES is about 220 nucleotides in length.

As mentioned above, the methods of making circular RNA described herein employ a Group I maturase or Group II maturase. The Group I or Group II maturase is paired with a Group I or a Group II intron, respectively. In this context. “paired” refers to a maturase that splices, or assists in splicing, a particular Group I or Group II intron. Table 2 provides exemplary Group I or II maturases with their Group I or II intron pairs, all of which are contemplated as within the scope of the disclosure. Also included are homologs of the Group I or Group II maturases described herein.

In some embodiments, the Group I intron is an Aspergillus nidulans COB1 intron and its paired maturase is an Aspergillus nidulans I-AniI maturase. The term “I-AniI maturase” includes the polypeptide of SEQ ID NO:3 or a polypeptide sequence having at or greater than at or greater than about 50%, at or greater than about 75%, at or greater than about 80%, at or greater than about 85%, at or greater than about 90%, at or greater than about 95%, or at or greater than about 98% identity with the polypeptide of SEQ ID NO:3, or a polypeptide comprising a portion of the polypeptide of SEQ ID NO:3. In some embodiments, the I-AniI maturase is N-terminally truncated in manner that preserves the maturase domain function. In some aspects, about 5-10 amino acids preceding the maturase domain are retained in the N-terminally truncated I-AniI maturase. In some embodiments, the I-AniI maturase is a polypeptide of SEQ ID NO:4 or a polypeptide sequence having at or greater than at or greater than about 50%, at or greater than about 75%, at or greater than about 80%, at or greater than about 85%, at or greater than about 90%, at or greater than about 95%, or at or greater than about 98% identity with the polypeptide of SEQ ID NO:4, or a polypeptide comprising a portion of the polypeptide of SEQ ID NO:4.

In some embodiments, the 3′ Group I intron sequence is or comprises SEQ ID NO:27 and the 5′ Group I intron sequence is or comprises SEQ ID NO:28. In some embodiments, the 3′ Group I intron sequence is or comprises SEQ ID NO:27 and the 5′ Group I intron sequence is or comprises SEQ ID NO:29. In some embodiments, the 3′ Group I intron sequence is or comprises SEQ ID NO:30 and the 5′ Group I intron sequence is or comprises SEQ ID NO:31. In some embodiments, the 3′ Group I intron sequence is or comprises SEQ ID NO:32 and the 5′ Group I intron sequence is or comprises SEQ ID NO:33. In some embodiments, the 3′ Group I intron sequence is or comprises SEQ ID NO:34 and the 5′ Group I intron sequence is or comprises SEQ ID NO:35.

In some embodiments, the Group II intron is an Oryza sativa lysine tRNA-K (UUU) intron and its paired maturase is an Oryza saliva maturase K. The term “maturase K” includes the polypeptide encoded by SEQ ID NO:1 or a polypeptide sequence having at or greater than about 50%, at or greater than about 75%, at or greater than about 80%, at or greater than about 85%, at or greater than about 90%, at or greater than about 95%, or at or greater than about 98% identity with the polypeptide encoded by SEQ ID NO:1, or a polypeptide comprising a portion of the polypeptide encoded by SEQ ID NO: 1. In some embodiments, the maturase is cyt-19 from N. Crassa and the intron is the aI5γ and bI1 group II intron from yeast. The term “cyt-19” includes the polypeptide of SEQ ID NO:2 or a polypeptide sequence having at or greater than at or greater than about 50%, at or greater than about 75%, at or greater than about 80%, at or greater than about 85%, at or greater than about 90%, at or greater than about 95%, or at or greater than about 98% identity with the polypeptide of SEQ ID NO:2, or a polypeptide comprising a portion of the polypeptide of SEQ ID NO:2. In some embodiments, the Group II intron is a Lactococcus lactis LI.LtrB intron and its paired maturase is a Lactococcus lactis LtrA maturase. The term “LtrA maturase” includes the polypeptide of SEQ ID NO:15 or a polypeptide sequence having at or greater than at or greater than about 50%, at or greater than about 75%, at or greater than about 80%, at or greater than about 85%, at or greater than about 90%, at or greater than about 95%, or at or greater than about 98% identity with the polypeptide of SEQ ID NO:15, or a polypeptide comprising a portion of the polypeptide of SEQ ID NO:15. In some embodiments, the L1.LtrB intron comprises or is SEQ ID NO:45.

In some embodiments, the Group II intron is a Lactococcus lactis LtrB intron and its paired maturase is a Lactococcus lactis LtrB maturase. In some aspects, the intron is nad1 i4 and the paired maturase is a MatR maturase. The term “MatR maturase” includes the polypeptide of SEQ ID NO:16 or a polypeptide sequence having at or greater than at or greater than about 50%, at or greater than about 75%, at or greater than about 80%, at or greater than about 85%, at or greater than about 90%, at or greater than about 95%, or at or greater than about 98% identity with the polypeptide of SEQ ID NO:16, or a polypeptide comprising a portion of the polypeptide of SEQ ID NO:16. In other aspects, the intron is a IIA intron aI2 and the paired maturase is MSS116. The term “MSS116” includes the polypeptide of SEQ ID NO: 17 or a polypeptide sequence having at or greater than at or greater than about 50%, at or greater than about 75%, at or greater than about 80%, at or greater than about 85%, at or greater than about 90%, at or greater than about 95%, or at or greater than about 98% identity with the polypeptide of SEQ ID NO: 17, or a polypeptide comprising a portion of the polypeptide of SEQ ID NO: 17. In still other aspects, the Group I or Group II intron and the paired maturase is as described in Table 2, or homologs thereof.

It should be understood that the Group I or Group II intron and its respective paired maturase can be from different species. It should also be understood that the maturase can be mutated at one or more amino acids to reduce or eliminate DNA endonuclease activity and/or increase splicing activity. The maturase can also be N-terminally truncated. In some embodiments, the maturase is mutated to increase its solubility.

Inclusion of the maturase and the maturase intron pairs in the present invention allows for milder reaction conditions and/or improved circular RNA yield. Accordingly, included herein are methods of making a circular RNA wherein the maturase polypeptide and the precursor RNA are contacted under conditions comprising a temperature of between 20° C. and 45° C. In some embodiments, the maturase polypeptide and the precursor RNA are contacted under conditions comprising a temperature of about 20° C. In some embodiments, the maturase polypeptide and the precursor RNA are contacted under conditions comprising a temperature of about 25° C. In some embodiments, the maturase polypeptide and the precursor RNA are contacted under conditions comprising a temperature of about 30° C. In some embodiments, the maturase polypeptide and the precursor RNA are contacted under conditions comprising a temperature of about 35° C. In some embodiments, the maturase polypeptide and the precursor RNA are contacted under conditions comprising a temperature of about 37° C. In some embodiments, the maturase polypeptide and the precursor RNA are contacted under conditions comprising a temperature of about 42° C.

In some aspects, the maturase polypeptide and the precursor RNA are contacted for approximately 30 minutes, approximately one hour, approximately two hours, approximately three hours, or approximately four hours. In some embodiments, the maturase polypeptide and the precursor RNA are contacted for approximately two hours. In some embodiments, the maturase polypeptide and the precursor RNA are contacted for approximately three hours. In some embodiments, the maturase polypeptide and the precursor RNA are contacted for approximately four hours.

In other or further embodiments, the maturase polypeptide and the precursor RNA are contacted under conditions comprising a magnesium concentration of between 100 micromolar and 25 millimolar. In some embodiments, the magnesium concentration is about 100 micromolar. In some embodiments, the magnesium concentration is about 75 micromolar. In some embodiments, the magnesium concentration is about 50 micromolar. In some embodiments, the magnesium concentration is about 25 micromolar.

Also included herein are methods of making circular RNA wherein the vector further includes two or more complementarity sequences. The present disclosure therefore includes a method of making circular RNA comprising:
a. transcribing a vector to form a precursor RNA, wherein the vector comprises operably linked elements ordered as follows:
   i. a 5′ complementarity sequence,
   ii. a 3′ Group I intron or 3′ Group II intron sequence containing a 3′ splice site dinucleotide,
   iii. a first non-coding sequence,
   iv. a corresponding 5′ Group I intron or 5′ Group II intron sequence containing a 5′ splice site dinucleotide, and
   v. a 3′ complementarity sequence; and
b. contacting the precursor RNA with a paired Group I or Group II maturase polypeptide to allow formation of the circular RNA.

The present invention also includes a method of making circular RNA comprising:
a) transcribing a vector to form a precursor RNA, wherein the vector comprises operably linked elements ordered as follows:
   i. a 3′ Group I intron or 3′ Group II intron sequence containing a 3′ splice site dinucleotide.
   ii. a first non-coding sequence,
   iii. a 5′ complementanty sequence,
   iv. a 3′ complementarity sequence;
   v. a corresponding 5′ Group I intron or 5′ Group II intron sequence containing a 5′ splice site dinucleotide, and
b) contacting the precursor RNA with a paired Group I or Group II maturase polypeptide to allow formation of the circular RNA.

In some methods, the vector further comprises an internal ribosome entry site (IRES) and a protein coding sequence and a second non-coding sequence, in that order, between the elements of a.iii. and a. iv.

It is to be understood that the 5′ and 3′ complementarity sequences are DNA sequences that are sufficiently complementary to one another to bind to one another, for example, on the basis of A-T complementarity and C-G complementarity. Each element of the vector or the transcribing or contacting steps in these embodiments can be any of those described herein. In some embodiments, the complementarity sequences are between 25 and 60 nucleotides in length. In some embodiments, the 5′ complementarity sequence comprises or is SEQ ID NO:37 and the 3′ complementarity sequence comprises or is SEQ ID NO:41.

In addition to methods of making circular RNAs, provided herein are circular RNAs made according to these methods and pharmaceutical compositions comprising the circular RNAs. As one example, the circular RNAs described herein can serve as virus-like protein vaccines for prevention of infections such as COVID-19. In some embodiments, the circular RNA comprises between about 300 and 12,000 nucleotides, between about 1,000 and 10,000 nucleotides, about 5,000 and 8,000 nucleotides, about 8,000 and 12,000 nucleotides, or about 10,000 and 12,000 nucleotides. The pharmaceutical compositions can include a pharmaceutically acceptable carrier or nanocarrier for administration of the pharmaceutical composition to a subject. Examples of pharmaceutically acceptable carriers and nanocarriers are provided herein. In some embodiments, the pharmaceutically acceptable nanocarrier is a lipid nanoparticle, a lipid, a lipid polymer, a lipo-polymeric hybrid, an exosome or a leukosome.

Also provided herein are methods of isolating the circular RNA described herein. Any appropriate method may be used for such isolation including the use of solid phase extraction (SPE) and size exclusion chromatography. In some embodiments, the method of isolating a circular RNA described herein comprises
a. obtaining a mixture of linear RNA and circular RNA created by any of the methods described herein;
b. contacting the mixture with one or more fixed 5′ binding proteins and one or more fixed 3′ binding proteins; and
c. isolating the circular RNA.

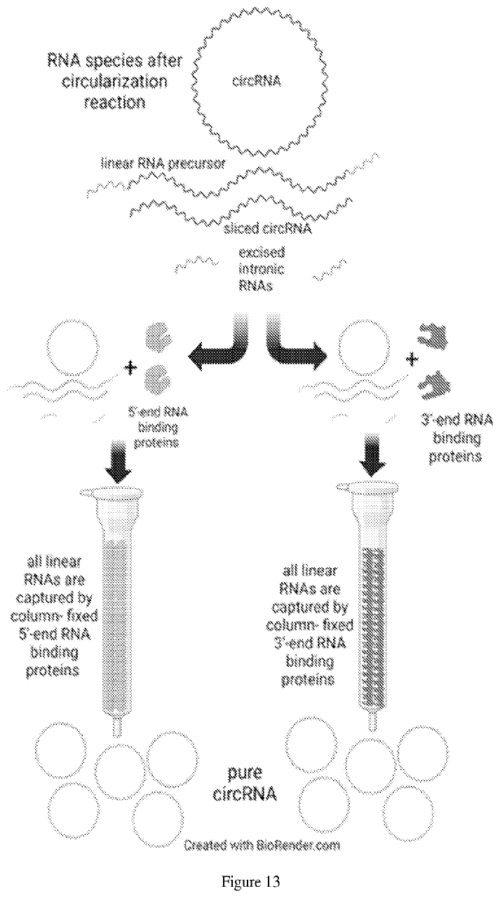

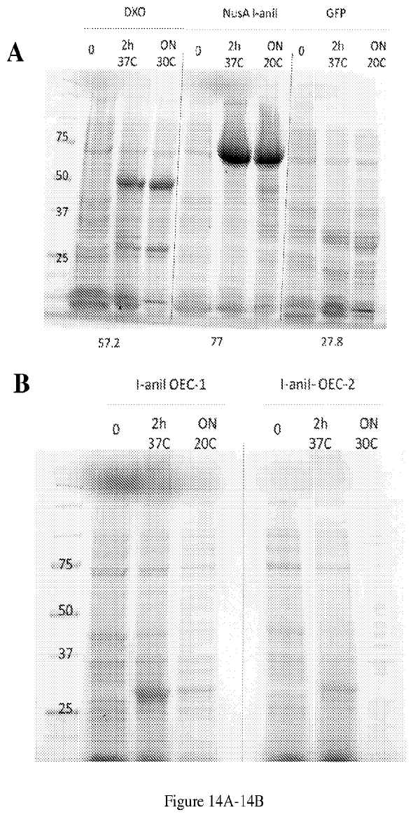

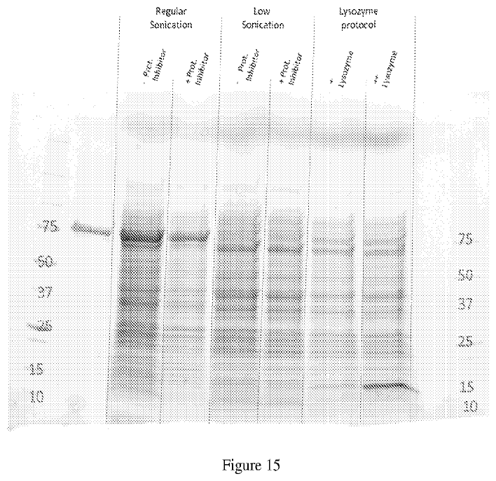

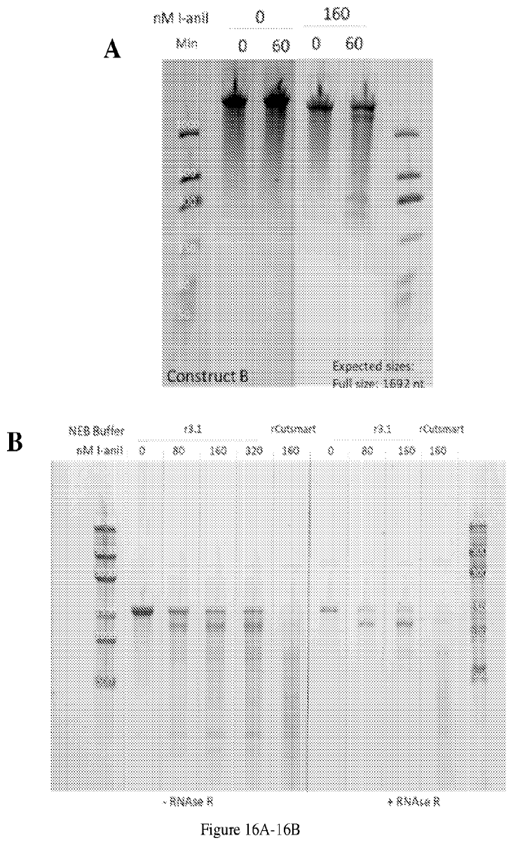

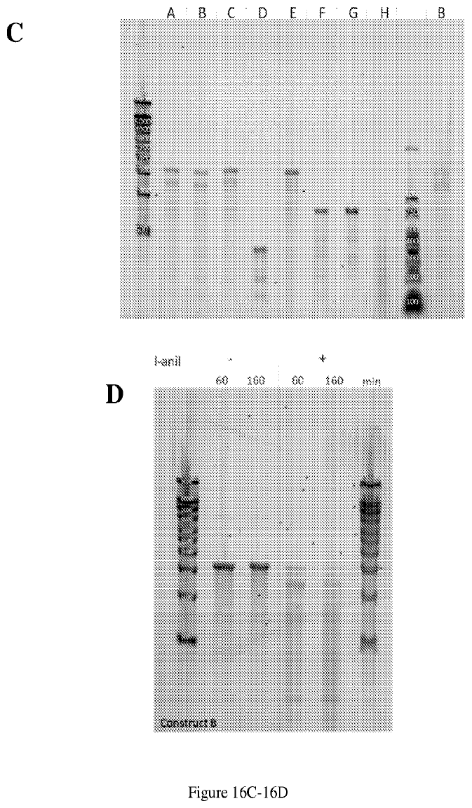

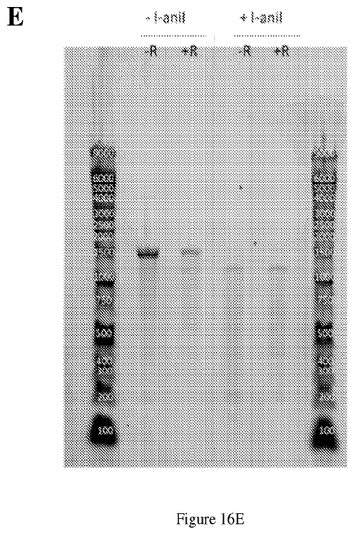

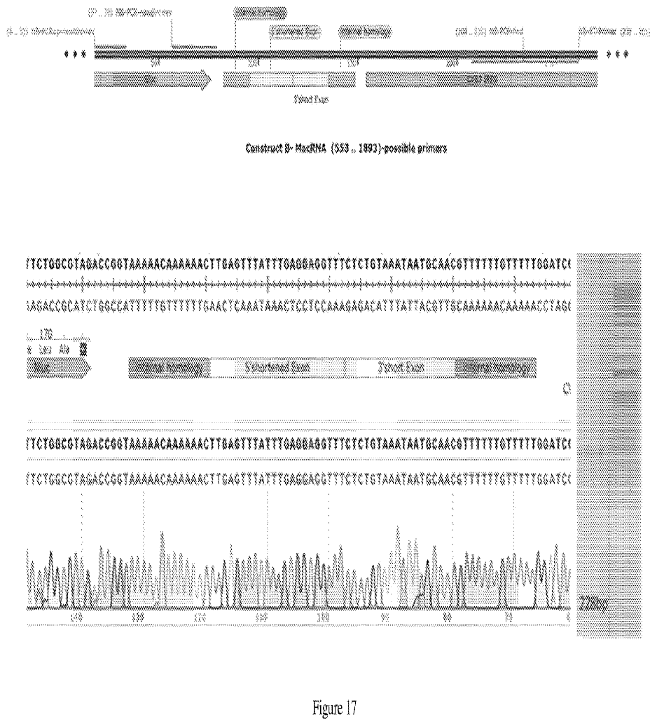

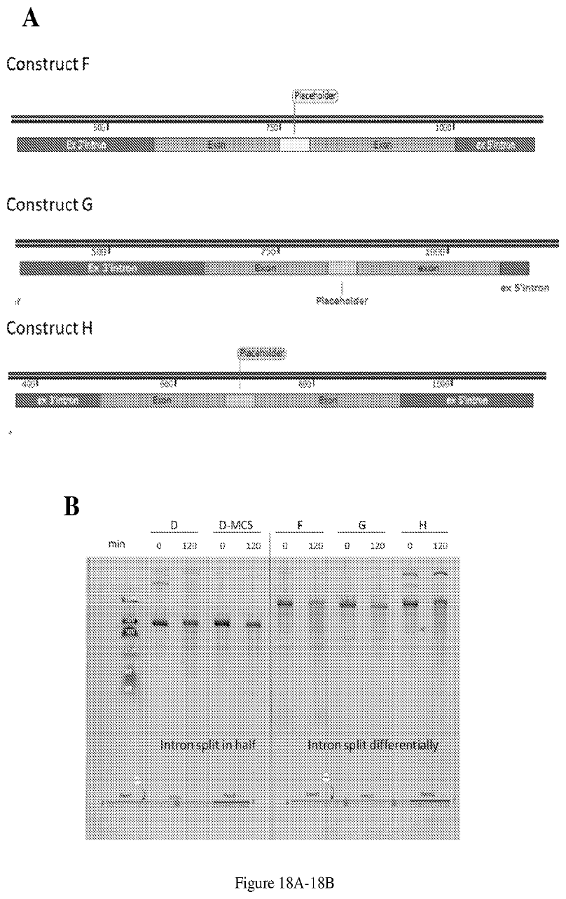

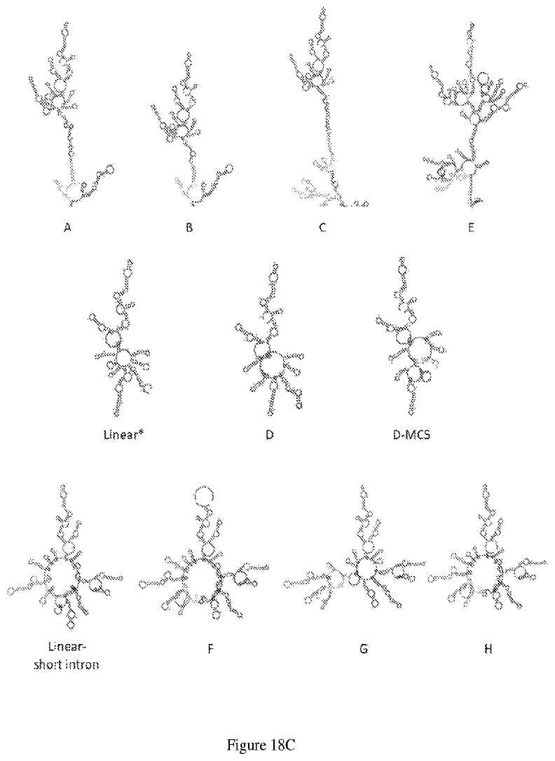

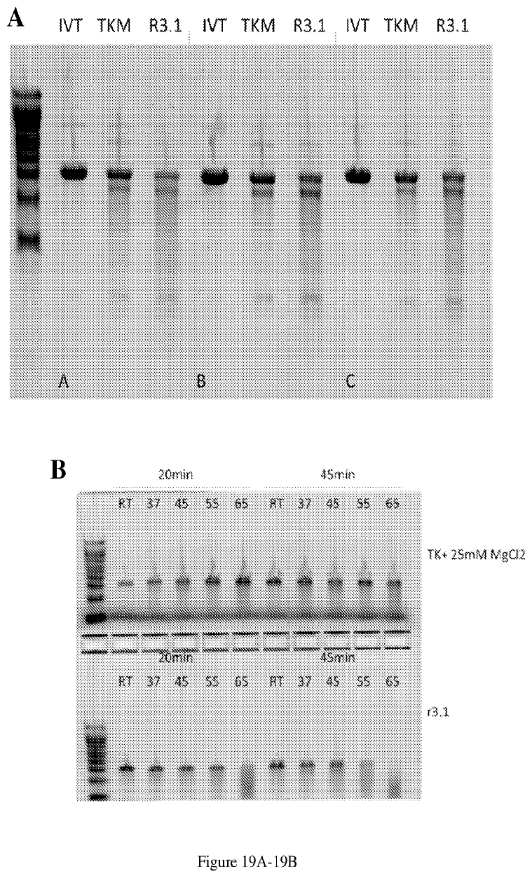

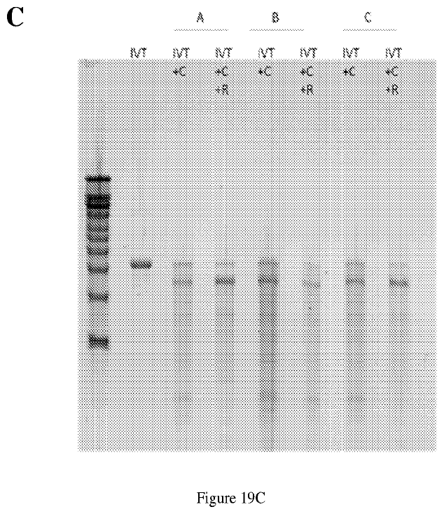

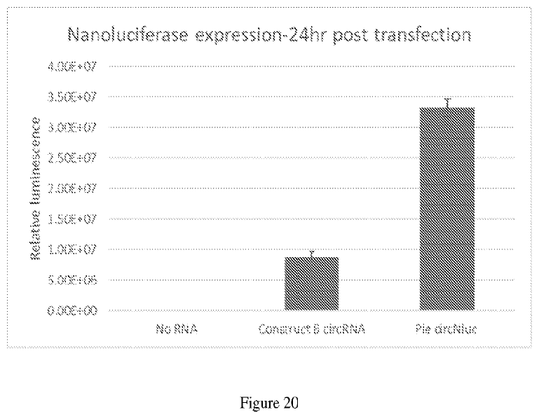

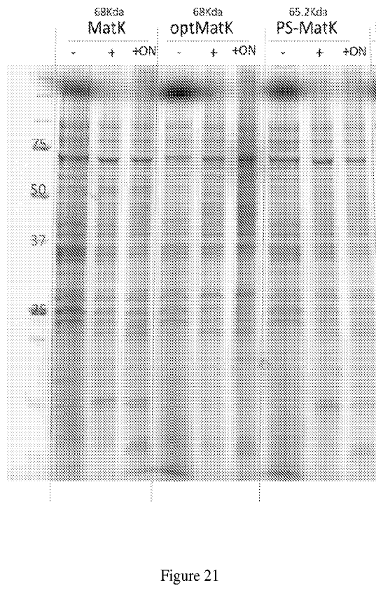

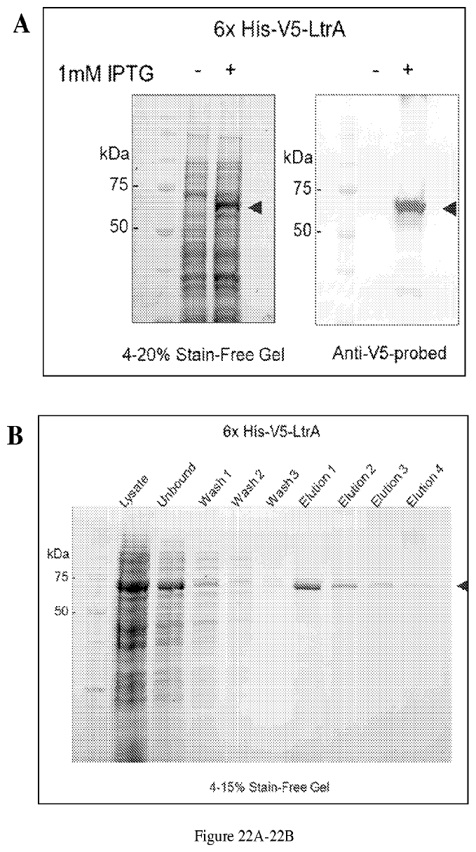

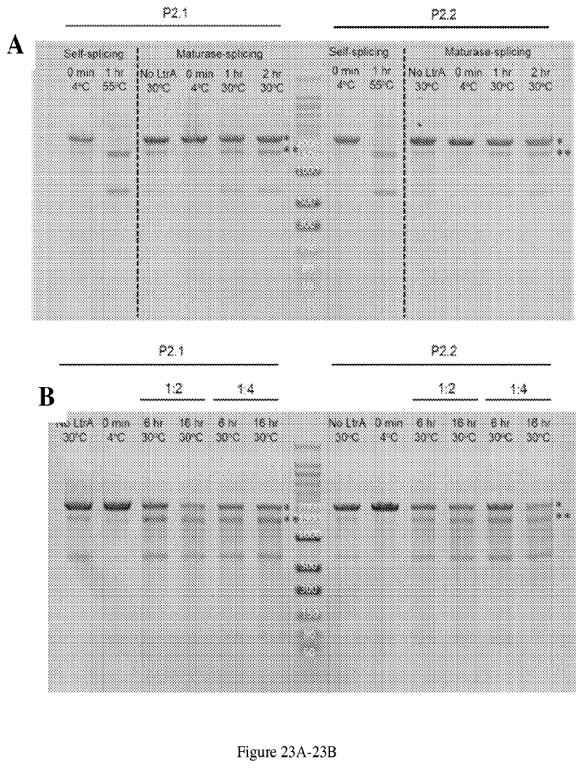

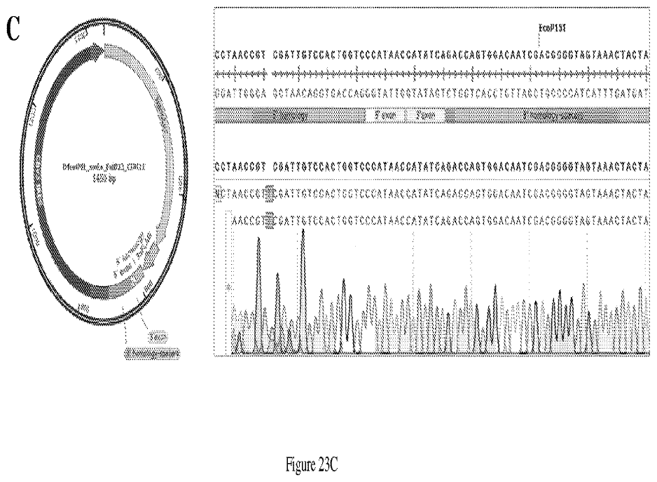

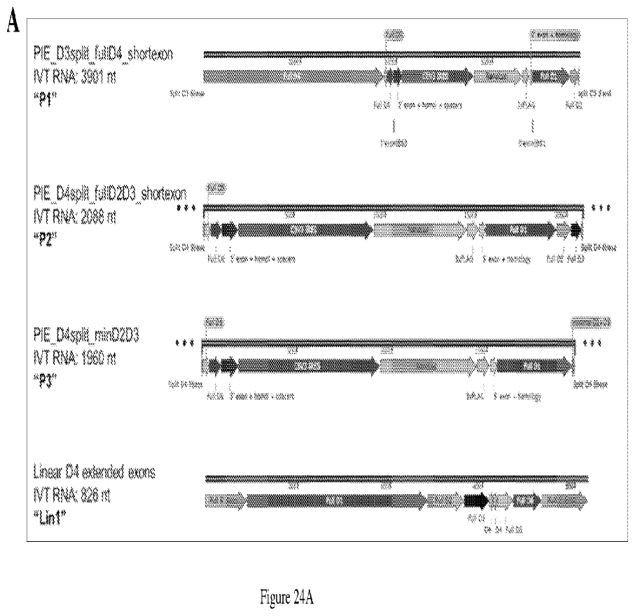

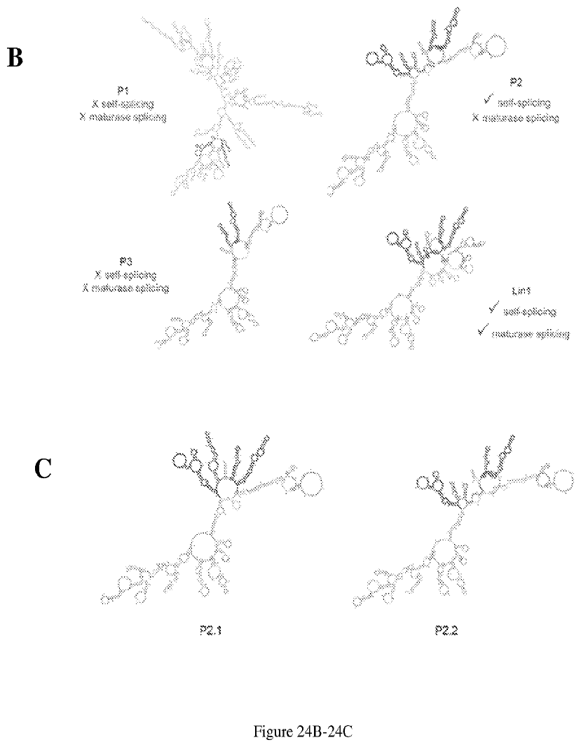

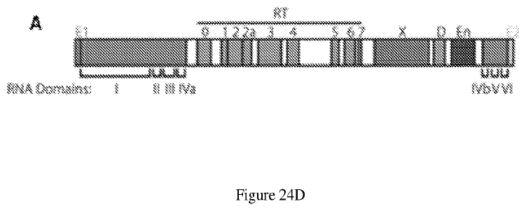

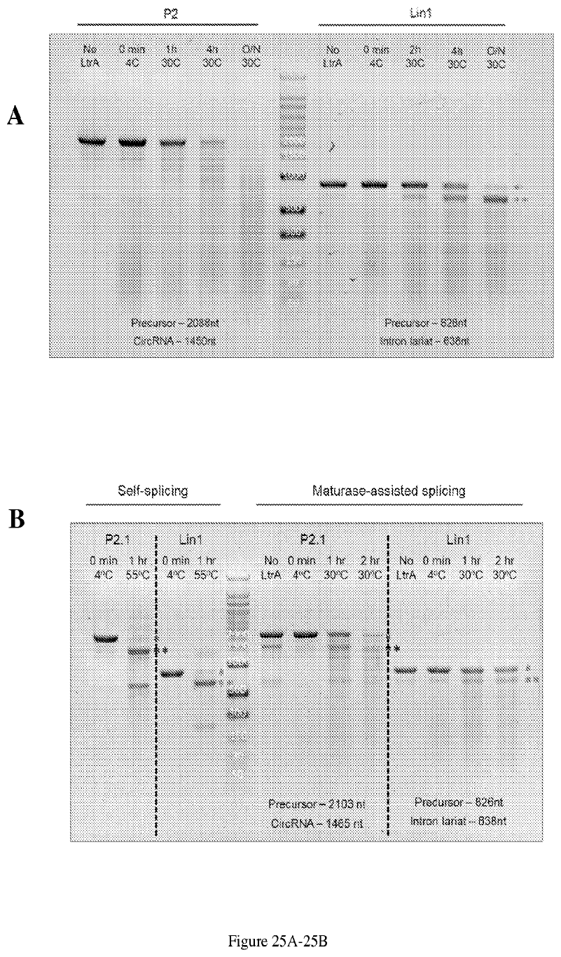

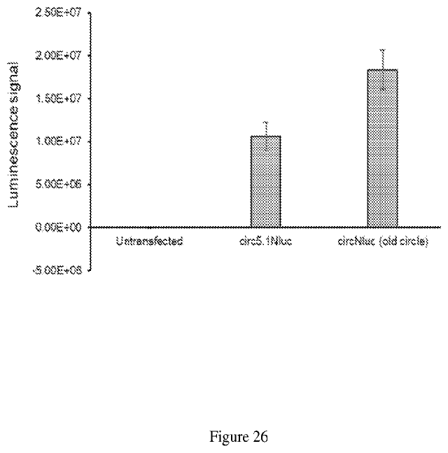

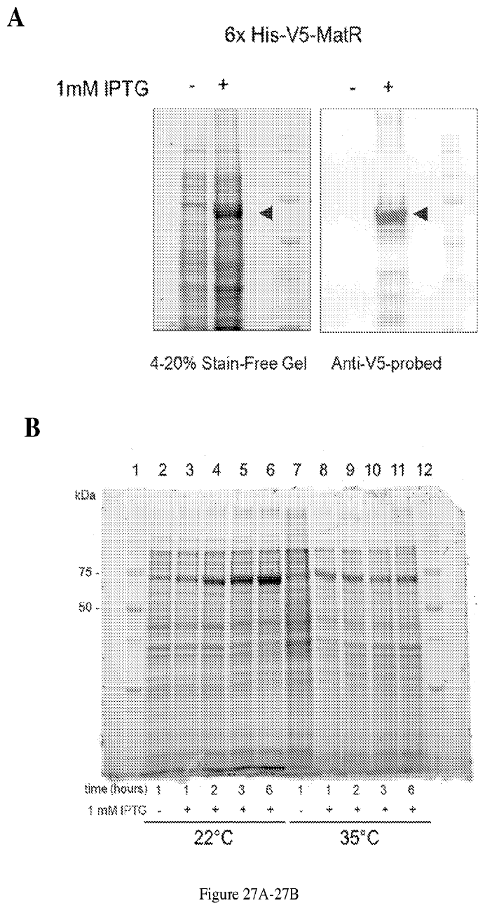

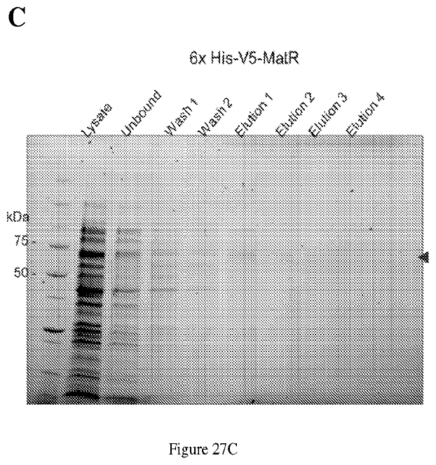

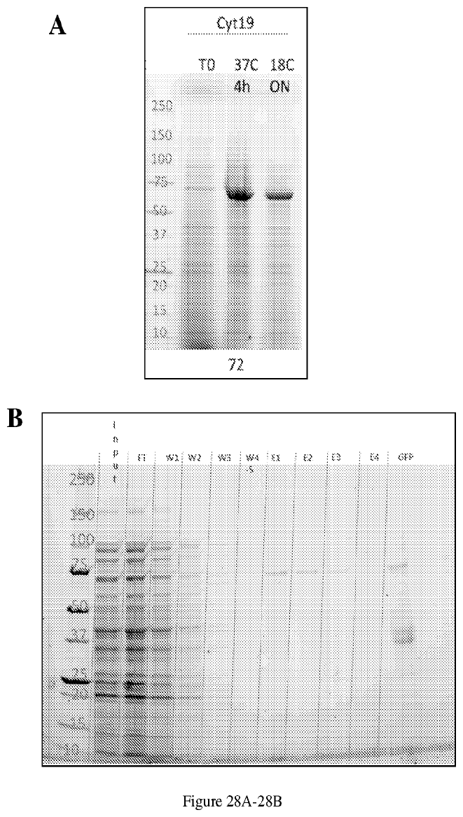

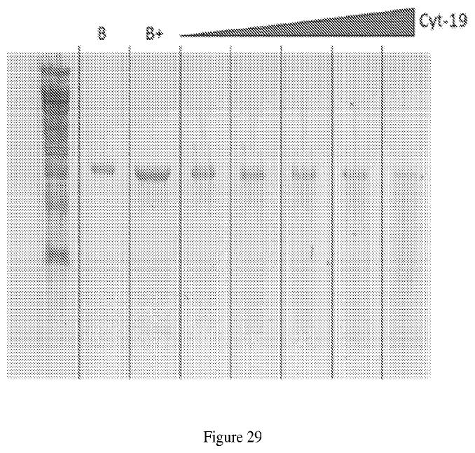

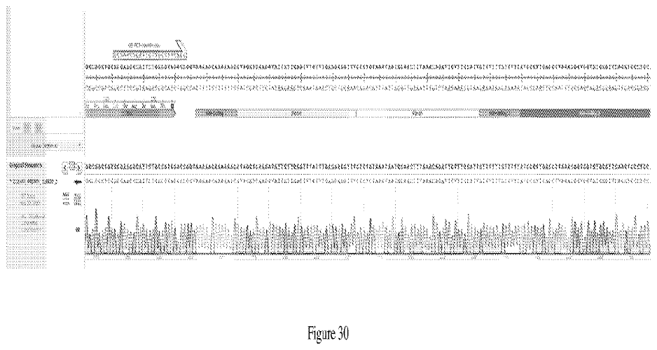

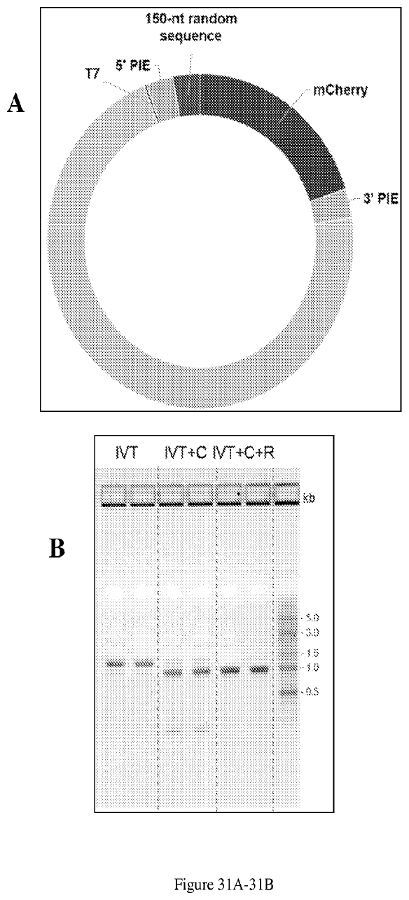

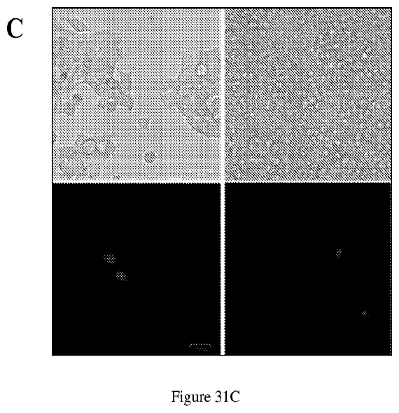

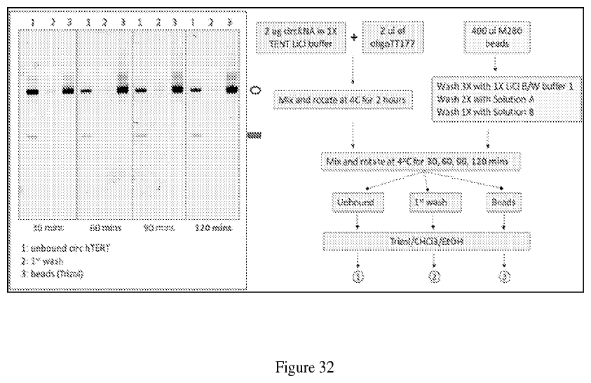

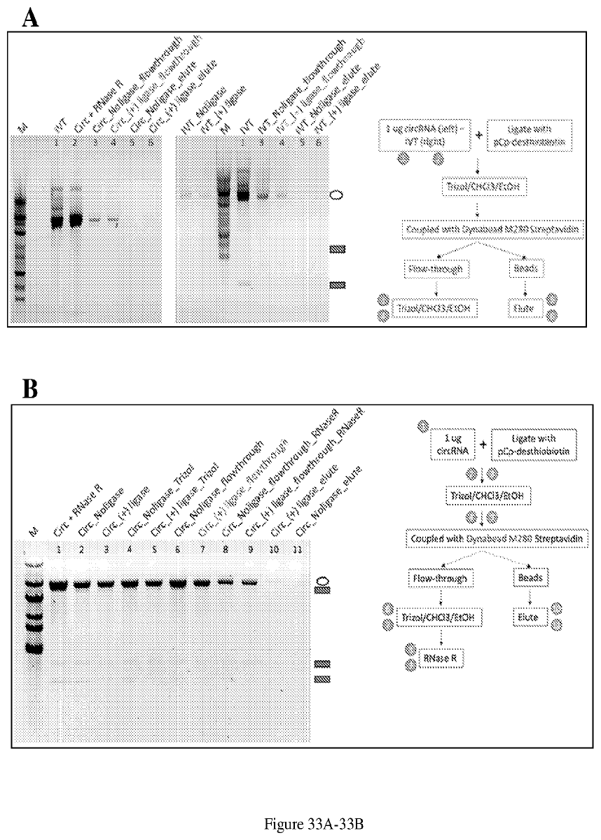

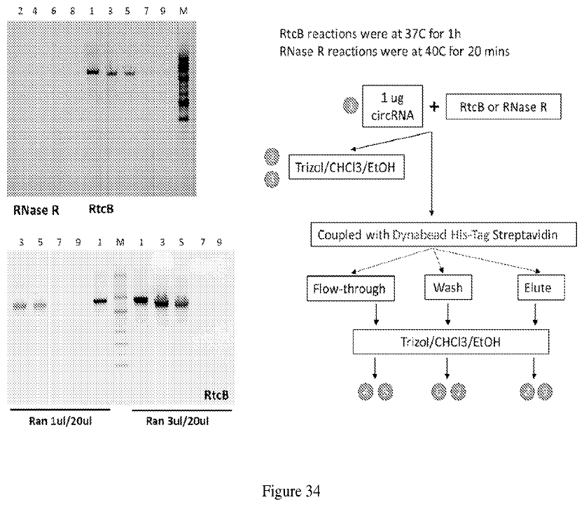

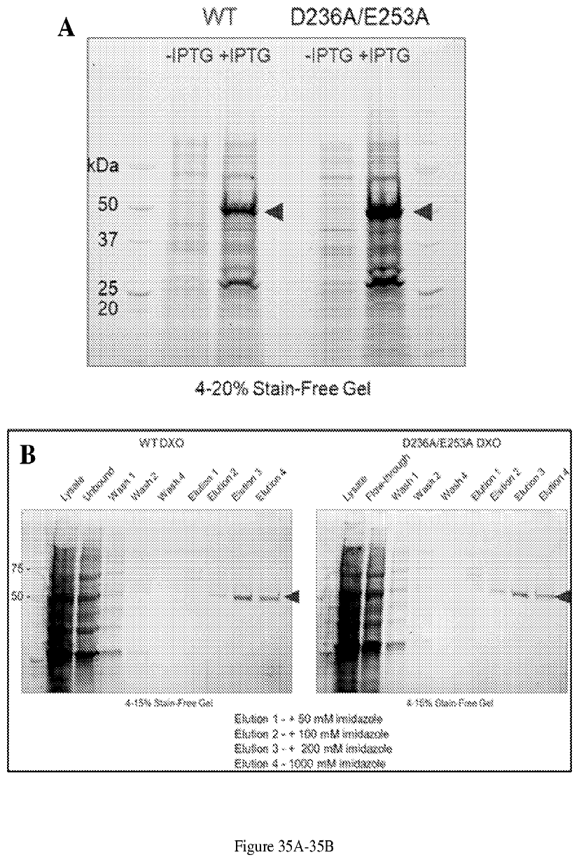

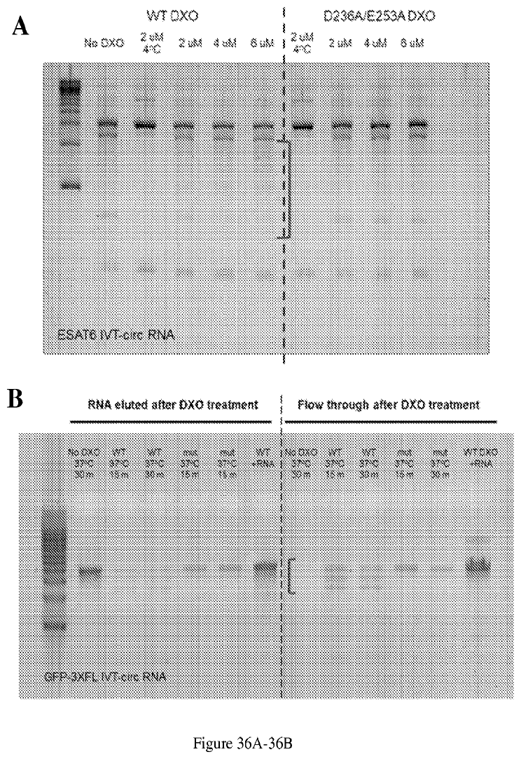

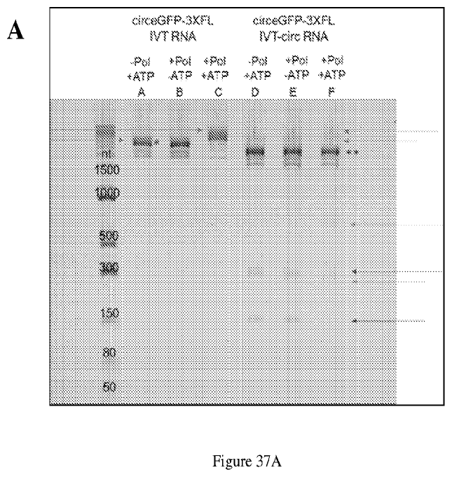

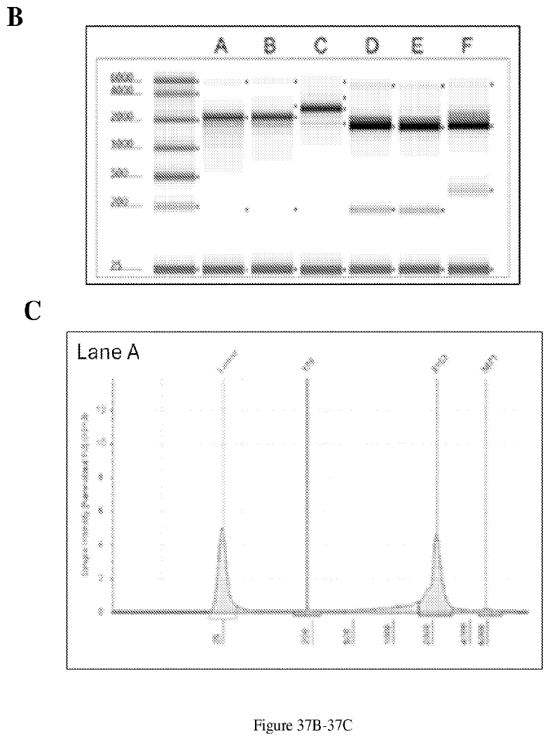

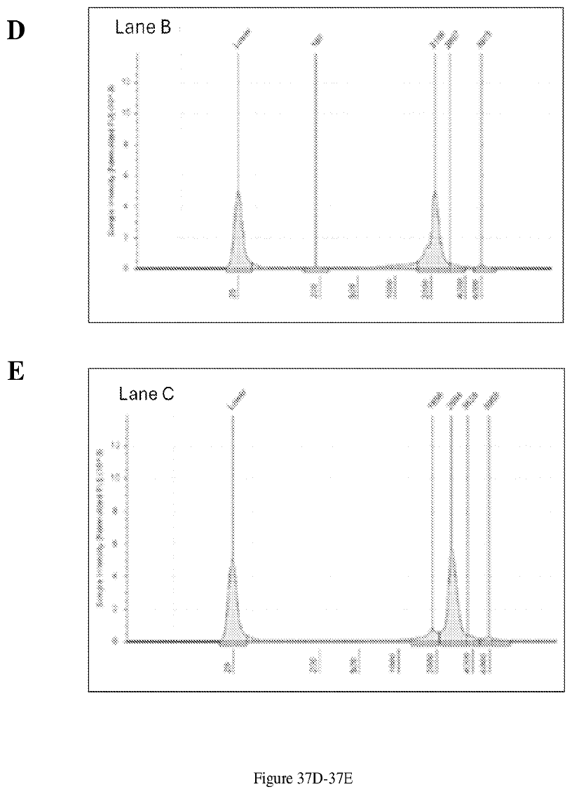

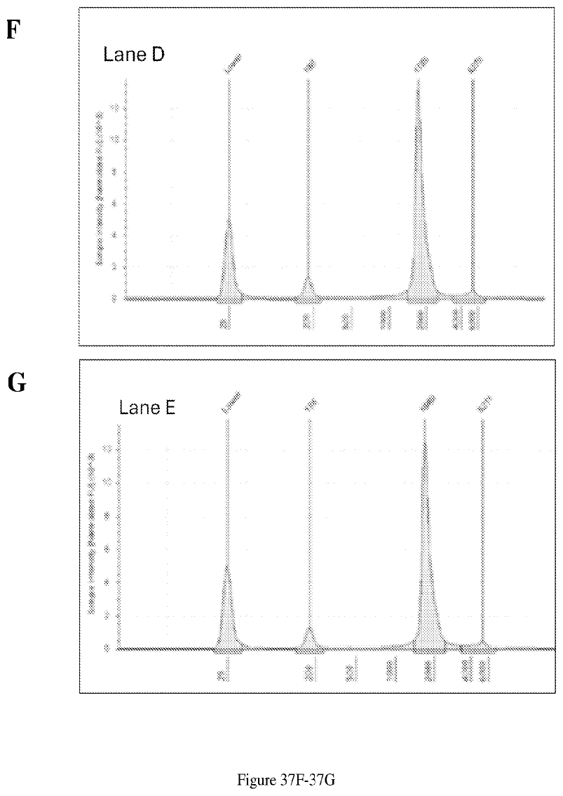

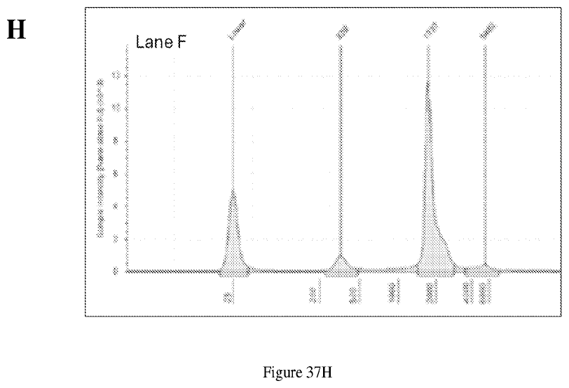

FIG. 13 shows a general schematic of one such method. The 5′ and 3′ binding proteins can be any that bind to 5′ and 3′ RNA ends. In some embodiments, the 5′ binding protein is a Saccharomyces cerevisiae DXO 5′ binding protein, or a homolog thereof. The term “DXO” includes the polypeptide encoded by SEQ ID NO:5 or a polypeptide sequence having at or greater than about 50%, at or greater than about 75%, at or greater than about 80%, at or greater than about 85%, at or greater than about 90%, at or greater than about 95%, or at or greater than about 98% identity with the polypeptide encoded by SEQ ID NO:5, or a polypeptide comprising a portion of the polypeptide encoded by SEQ ID NO:5. In some embodiments, the DXO 5′ binding protein is inactivated. In some embodiments, the 5′ binding protein is an inactivated RNase R, or a homolog thereof. In some embodiments, the 5′ or 3′ binding protein has a mutation that increases or changes its temperature or pH sensitivity. In some embodiments, the RNA binding protein is mutated, truncated, or fused to another protein domain to increase its solubility and/or RNA binding affinity.

In other or further embodiments, the method of isolating a circular RNA described herein comprises
a. obtaining a mixture of linear RNA and circular RNA created by any of the methods described herein;
b. using poly(A) polymerase to add a biotinylated adenosine to the linear RNA to create a biotinylated linear RNA; and
c. isolating the circular RNA by removing the biotinylated linear RNA using a fixed streptavidin.

In other or further embodiments, the method of isolating a circular RNA described herein comprises
a. obtaining a mixture of linear RNA and circular RNA created by any of the methods described herein;
b. using RtcB RNA ligase to modify the linear RNA:
c. contacting the mixture with a fixed His-Tag streptavidin that binds the modified linear RNA; and
d. isolating the circular RNA from the flow through.

In other or further embodiments, the method of isolating a circular RNA described herein comprises
a. obtaining a mixture of linear RNA and circular RNA created by any of the methods described herein;
b. contacting the mixture with a polynucleotide specific for a circularization junction of the circular RNA bound to biotin to create a second mixture;
c. contacting the second mixture with a fixed streptavidin;
d. removing the linear RNA; and
e. isolating the circular RNA by releasing the circular RNA from the one or more fixed polynucleotides.

In some aspects, the polynucleotide specific for the circularization junction of the circular RNA is a locked nucleic acid (LNA) and the polynucleotide is complementary to a sequence in the circularization junction of the circular RNA.

It should be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims. All patents, patent applications, and publications referenced herein are incorporated by reference in their entirety for all purposes.

### EXAMPLES

### Example 1. CircRNA Performs Better than Linear RNA

The performance of circRNA versus linear was compared with the expression of green fluorescence protein (GFP). Briefly circRNA and linear RNA were transfected into HEK293, and fluorescence intensity was monitored using a live cell imaging system (Incucyte, Sartorius). It was observed that GFP expression from circRNA lasts longer and was still increasing after 48 hours, whereas GFP expression from linear RNA starts decreasing after 24 hours. See FIG. 2.

### Example 2. A.n. Cob1 Intron Pre-RNA Splices Successfully In Vitro and can be Visualized with an Agarose Gel

Method: RNA for was made by in vitro transcription using the HiScribe T7 High Yield RNA Synthesis Kit (new England Biolabs) starting with linearized plasmid. This was followed by purification using the Monarch RNA cleanup 500ug (New England Biolabs). RNA was preincubated in 2X TN buffer (100 mM Tris-HCl pH 7.5, 200 mM NaCl. 300 mM MgCl₂) at 37C for 20 min. 2xGTP solution (2 mM rGTP (Promega)) was then added to initiate splicing. Aliquots were collected at the times indicated and the reaction was column-purified using Monarch RNA cleanup columns. The RNA (denatured with 50% Formamide, 70° C. for 3 min) was visualized using an e-gel system.

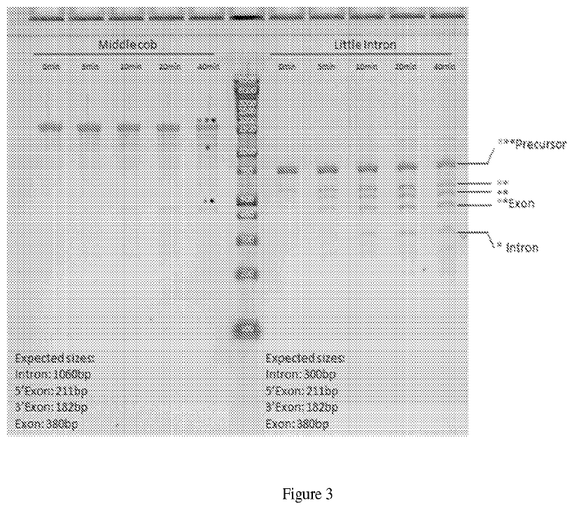

Results: The linear RNA can self-splice at 37° C. using these conditions in a solution of 50 mM Tris-HCL pH 7.5; 100 mM NaCl, 150 mM MgCl2 and 1 mM GTP. The precursor RNA decreases over time and the linked exon and free introns were visualized as well as intermediate of the splicing reaction. See FIG. 3.

After validating that the linear RNA can self-splice in vitro under high magnesium conditions, the focus was shifted to maturase-assisted splicing. For a successful macRNA production, it was required to purify the maturase, and create a permutated intron/exon (PIE) version from the RNA to be spliced.

### Example 3. I-AniI Purification

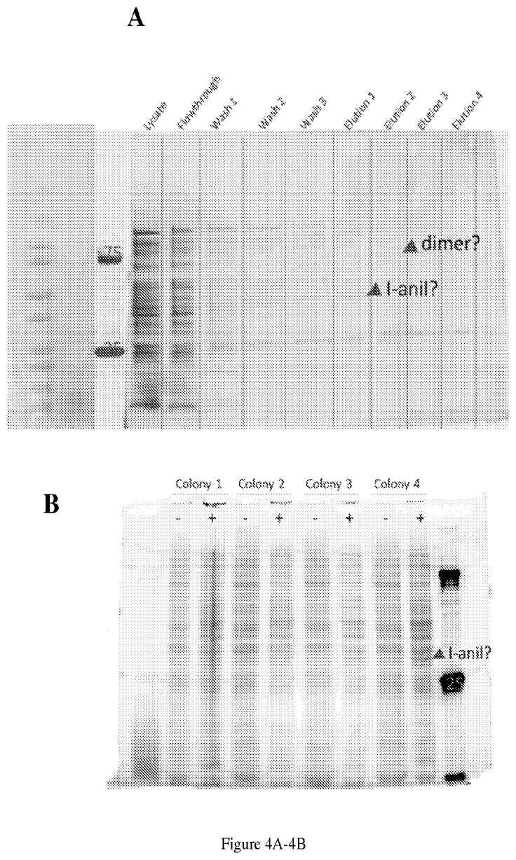

The I-aniI expression vector was designed using their codon optimization tool, cloned into their premier expression vector (optimized for expression in E. coli) and purchased from Geneart (Thermofisher) as a complete plasmid. The plasmid was first transformed into the custom cell line for the plasmid BL21-DE3-star (Thermofisher). Colonies were used to inoculate a 5 ml culture in LB+Ampicillin (amp). The next day, 1 ml of culture was used to inoculate a 50 ml culture LB+amp and OD is followed until it reaches 0.6-0.8 for log phase. IPTG at a final concentration of 1 mM was added to the culture was incubated ON at a reduced temperature (30° C.). An expression test was performed by running an SDS gel of the lysed bacterial pellet. See FIG. 4.

The initial attempts show limited expression of the I-aniI maturase using this plasmid. We are troubleshooting by varying the media used (Superior broth (AthenaES) used to allow the bacteria with extra nutrients and reaches a higher density); reducing the temperature of the incubation to 18° C. to allow the protein to fold correctly; changing the stock of IPTG used as well as having a positive control. Possible problems could be the codon optimization (a fast translation can lead to a misfolded protein), trying a different plasmid expression system etc. See FIG. 4.

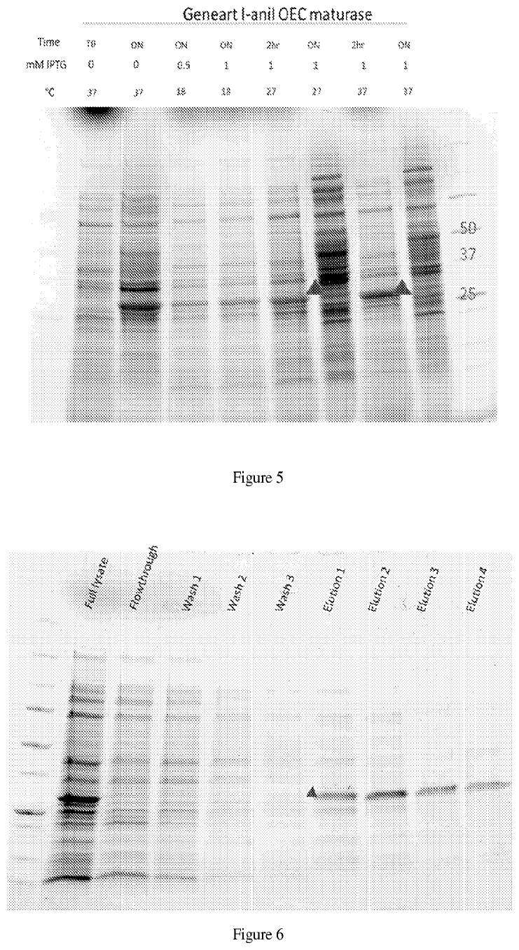

In subsequent attempts to express the I-aniI maturase, the I-aniI was subcloned to a new expression backbone from addgene by swapping the ORF from GFP to the maturase. A new full size open reading frame for the I-aniI was also obtained from geneart. All constructs were tested using the same method described above (superior broth, induction when log phase is achieved with 0.5 mM or 1 mM IPTG). Different incubation temperatures were tested and different time points. The expression of the maturase with the new full size open reading frame can be seen in FIG. 5.

The maturase I-aniI was purified as follows: pellet of bacteria was resuspended in lysis buffer (TxPBS, 100 μM PMSF, 250 mM NaCl. 5 mM Imidazole). After that, the mix was sonicated at 4° C. for 15 min (cycles of 15 sec ON, 15 sec OFF, high frequency). The solution was centrifuged at 4500 rpm for 40 min at 4° C., the supernatant was transferred to a new tube. His-Pur Resin (Thermofisher) was equilibrated with buffer (1XPBS, 10 mM imidazole). An equilibration buffer was added to the supernatant such as the volume obtained is equivalent to 3 beds of His-Pur resin. The solution was incubated with the equilibrated resin and rotated on an end-over-end shaker for 90 min at 4° C. The flowthrough was discarded, and 4 rounds of washes (Wash buffer: 1xPBS, 25 mM Imidazole, 100 mM NaCl) are done for the resin. Finally, the protein is eluted gradually by incubating the resin with the elution buffer (lx PBS, 250 mM imidazole, 100 mM NaCl). Samples were then run in an SDS page gel. Shown below are the different steps of purification, the elutions show a band (marked with an arrow) that corresponds to I-aniI. See FIG. 6.

### Example 4. Preserving Splicing Position and Folding of the Ribozyme is Critical for the Pie-Version

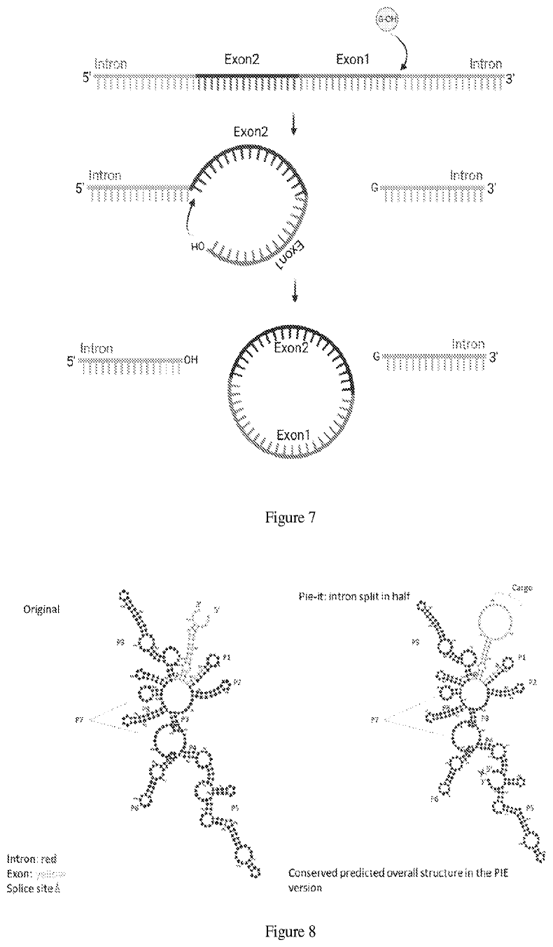

The ribozyme function heavily relies on its proper folding into a catalytically active form. A structure prediction approach was used to try to match the folding of the natural RNA to the synthetic pie version. RNAfold was used to predict the structures. As mentioned earlier, the natural ribozyme consists of an intron sandwiched between a 5′ and a 3′ exon. To hack the ribozyme into generating a circle, the position of the intron and exons is shuffled, and the intron is divided into two portions. See FIG. 7.

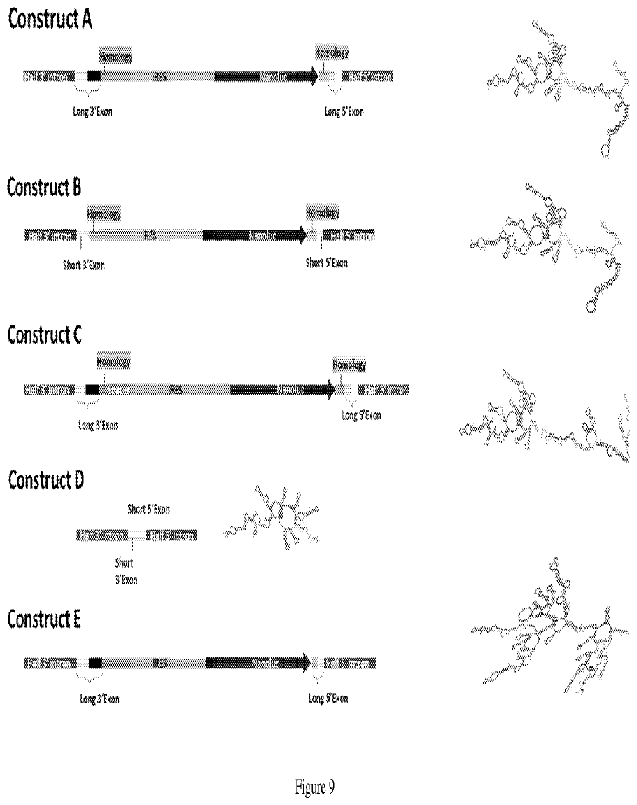

The structures of different introns splits were tested using RNAfold. It was determined that dividing the intron in half results in a very comparable structure with splicing sites arranged similarly. It was also determined that having a shorter 5′intron portion would still preserve the overall structure, the opposite (a longer 5′intron portion) results in a scrambled structure. See FIG. 8. Five (5) constructs, A through E, were generated with different layouts obtained though geneart. See FIG. 9. The selection of these constructs is explained in Table 1 below.

TABLE 1

| Construct | | |
|---|---|---|
| name | | |
| based | | |
| on A.n. | | |
| Cob1 | | |
| intron | Features | Reason to test |
| A | Intron split in half, | The intron is predicted to be |
| (SEQ ID | long exons, internal | folded correctly and separated |
| NO: 6) | homology arms (with | from the cargo |
| | IRES and Nanoluciferase) | (IRES + Nanoluciferase) |
| B | Intron split in half, | The intron is predicted to be |
| (SEQ ID | short exons, internal | folded corrected but is a little |
| NO: 7) | homology with (with | closer to the cargo. It should |
| | IRES and Nanoluciferase) | contain a smaller “scar” on the |
| | | final circle with smaller exons |
| C | Intron split in half, | A spacer sequence is added that |
| (SEQ ID | long exons, internal | might give more separation |
| NO: 8) | homology arms, spacer | between the cargo and the |
| | (with IRES and | introns |
| | Nanoluciferase) | |
| D | Intron split in half, short | The pie version, no cargo with |
| (SEQ ID | exons, (without IRES | short exons |
| NO: 9) | and Nanoluciferase) | |
| E | Intron split in half, long | This version presents issues of |
| (SEQ ID | exons, with (with IRES | folding and should not be |
| NO: 10) | and Nanoluciferase) | working; selected as negative |
| | | control for splicing. |
| F | Intron split with a | This version takes into account |
| (SEQ ID | rationale, long exons | that % of GC content superior to |
| NO: 11) | (without IRES and | 35% allows a better splicing |
| | Nanoluciferase) | efficiency |
| G | Intron split randomly, | This version is a random split of |
| (SEQ ID | long exons (without | the intron, with a short 3′end |
| NO: 12) | IRES and Nanoluciferase) | |
| H | Intron split randomly, | This version is a random split of |
| (SEQ ID | long exons (without | the intron, with a long 3′end |
| NO: 13) | IRES and Nanoluciferase) | |

### EXAMPLE 5. ADJUSTMENT OF SPLICING CONDITIONS

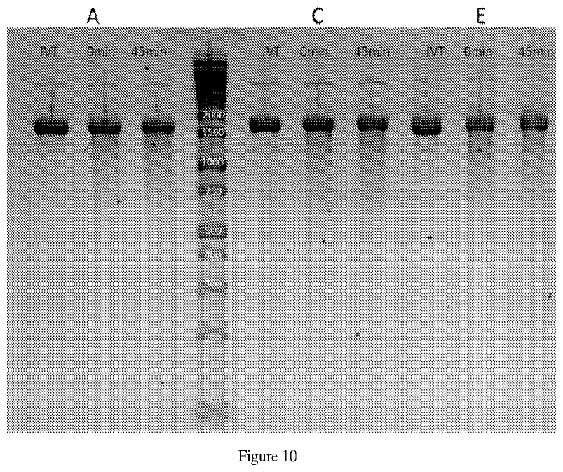

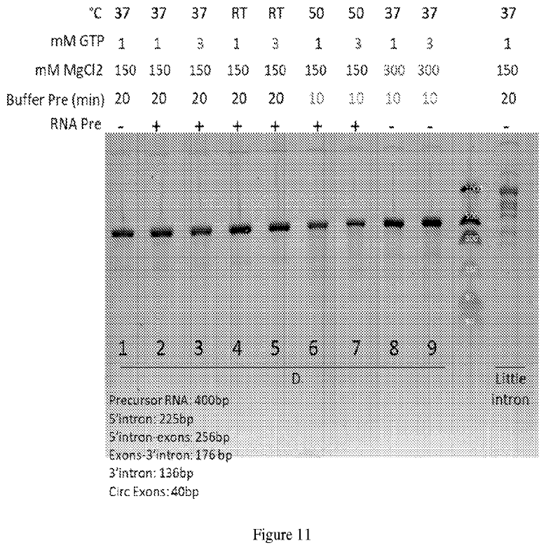

Using in vitro transcription (IVT), the RNA of each construct was obtained and tested for self-splicing using the same conditions used for the unshuffled RNA. It appears that a level of splicing is already occurring during or after IVT. A level of degradation also appears, possibly due to the high magnesium content. See FIG. 10. The concentration of magnesium will be highly reduced in presence of the maturase (from 150 mM to 5 mM final concentration) which will likely reduce the degradation of the RNA.

Multiple splicing conditions for construct D (no cargo) were tested and a difference in splicing was observed, especially when preincubating the RNA for 10 min instead of 20 min in the buffer before adding the splicing trigger (GTP). These steps will allow the adjustment of the splicing conditions when using the maturase. See FIG. 11.

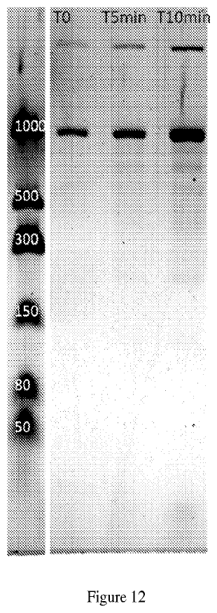

Constructs F, G, and H described in Table 1 were also developed. This was done as an attempt to improve upon splicing constructs A through E, which involved splitting the intron in half exactly, by using the shortened exons. Thus, for constructs F, G and H, the intron was split in 2 positions (generating a long or new short 5′end). Construct G and H were generated by selecting a random position for the intron to be split and the exons are the same size as the ones used on the little intron construct (SEQ ID NO: 14). Construct F was created to modulate the percentage of GC content, which may affect the splicing efficiency. As shown in FIG. 12, construct F seemed to be responsive to the splicing conditions (showing extra bands appearing overtime) and could potentially be the preferred construct.

In between the two exons, where the cargo is going to go, a placeholder (a small sequence adding multiple cloning sites) may be added. A spacer sequence having many AAATTT that could base-pair with the intron may also be added. Downstream of the whole sequence, a sap1 restriction site was added for future IVT.

### Example 6. Maturase-Intron Pairs

Along with the I-aniI maturase- A.n. Cob1 intron system, other maturases/intron pairs may be effective. Exemplary pairs are provided below in Table 2.

TABLE 2

| Maturase | Intron | Species |
|---|---|---|
| IaniI | COB1 | Aspergillus nidulans |
| MatK | trnK gene | Oryza sativa |
| | tRNAs (V-UAC, I-GAU, | |
| | A-UGC, and K-UUU) | |
| | ribosomal proteins | |
| | (rpl2 and rps12) | |
| | chloroplast ATPase | |
| | (atpF) | |
| LtrA | LtrA intron | Lactococcus lactis |
| LtrB | LtrB intron | Lactococcus lactis |
| LtrA | LI.LtrB | Lactococcus lactis |
| CYT-19 | al5γ and bI1 group | Yeast (intron) |
| | II intron (group II | Neurospora crassa (cyt- |
| | intron) | 19) |
| CYT-19 | IIA intron aI2 | Saccharomyces |
| | | cerevisiae |
| Mss116p | IIA intron aI2 | Saccharomyces |
| | | cerevisiae |
| MatR | nad1 i4 & various introns | Brassicaceae plant family |
| atnMat2 | single intron within | Plant |
| | cox2, nad1 intron2, | |
| | and nad7 intron2. | |
| Bl2, Bl3, Bl4 | Cob gene/CoX1 gene | Saccharomyces |
| | | cerevisiae |
| VibI5 | Cyt. b | V. inaequalis |
| AI4alpha | Cyt oxidase subunit 1 | Saccharomyces |
| (mutated) | | cerevisiae |
| Bl2 | Cyt. B | S. Capensis |
| COX1L1 | Cyt. Oxidase subunit I | S. pombe |

TABLE 3
List of IRES sequences to use as a test for circRNA specificity.

| Tag | Full name | IRES tested |
|---|---|---|
| A14 | circPPT19- | PPt19 |
| | mcherry | |
| A15 | circA1- | IRES00103 (RousSarV |
| | mcherry | IRES) |
| A16 | circA3- | IRES488 (AQP4 IRES) |
| | mcherry | |
| A17 | circA4- | IRES190 (HPV IRES) |
| | mcherry | |
| A18 | circA5- | IRES498 (LineORF1) |
| | mcherry | |
| A19 | circA7- | IRES485 (bag1) |
| | mcherry | |
| A20 | circA9- | IRES487 (ELG1) |
| | mcherry | |
| A21 | circA13- | RBM3 IRES |
| | mcherry | |
| A22 | circA14- | At1R IRES |
| | mcherry | |
| A23 | circA6- | IRES494 (LineORF1) |
| | mcherry | |
| A24 | circA8- | IRES476 (KMI1) |
| | mcherry | |
| A25 | circA15- | Pink-eared duck IRES |
| | mcherry | |
| A26 | circA16- | Caspase8 IRES |
| | mcherry | |
| A27 | circ-mcherry | CVB3 |
| A28 | circA2- | IRES240 (ELH) |
| | mcherry | |

Select Sequences:

SEQ ID NO: 6 Construct A; Underlined: Intronic sequence; Bolded:
Nanoluciferase; Bolded Underlined: CVB3 IRES; double underline: homology arms;
Italic: short exons; Italic underlined: extended exons.
ACGGGGAAAATCAGCAGGGGTGAGAAGTTTACATACTTCAGAAGCCTCTCAG
AGACTACATGCAGGAGATCTTATGTCGACATAAGATCATGATATAGTCCGAT
CAATAAAGAAATTTATTGCGTATAGTAAGAGGATTTAATATTTATATTAAATC
TGTAACTATCAACATAAATGCTCTGTAAATAATGCAACTTTAAACAGATTCTTTGC
ATTGTTTTTTGTTTTTGGATCCTTAAAACAGCCTGTGGGTTGATCCCACCCA
CAGGCCCATTGGGCGCTAGCACTCTGGTATCACGGTACCTTTGTGCGCC
TGTTTTATACCCCCTCCCCCAACTGTAACTTAGAAGTAACACACACCGAT
CAACAGTCAGCGTGGCACACCAGCCACGTTTTGATCAAGCACTTCTGTT
ACCCCGGACTGAGTATCAATAGACTGCTCACGCGGTTGAAGGAGAAAGC
GTTCGTTATCCGGCCAACTACTTCGAAAAACCTAGTAACACCGTGGAAG
TTGCAGAGTGTTTCGCTCAGCACTACCCCAGTGTAGATCAGGTCGATGA
GTCACCGCATTCCCCACGGGCGACCGTGGCGGTGGCTGCGTTGGCGGC
CTGCCCATGGGGAAACCCATGGGACGCTCTAATACAGACATGGTGCGAA
GAGTCTATTGAGCTAGTTGGTAGTCCTCCGGCCCCTGAATGCGGCTAAT
CCTAACTGCGGAGCACACACCCTCAAGCCAGAGGGCAGTGTGTCGTAAC
GGGCAACTCTGCAGCGGAACCGACTACTTTGGGTGTCCGTGTTTCATTT
TATTCCTATACTGGCTGCTTATGGTGACAATTGAGAGATCGTTACCATAT
AGCTATTGGATTGGCCATCCGGTGACTAATAGAGCTATTATATATCCCTT
TGTTGGGTTTATACCACTTAGCTTGAAAGAGGTTAAAACATTACAATTCA
TTGTTAAGTTGAATACAGCAAAATGGACGTCGTCTTCACACTCGAAGATT
TCGTTGGGGACTGGCGACAGACAGCCGGCTACAACCTGGACCAAGTCCT
TGAACAGGGAGGTGTGTCCAGTTTGTTTCAGAATCTCGGGGTGTCCGTA
ACTCCGATCCAAAGGATTGTCCTGAGCGGTGAAAATGGGCTGAAGATCG
ACATCCATGTCATCATCCCGTATGAAGGTCTGAGCGGCGACCAAATGGG
CCAGATCGAAAAAATTTTTAAGGTGGTGTACCCTGTGGATGATCATCACT
TTAAGGTGATCCTGCACTATGGCACACTGGTAATCGACGGGGTTACGCCGAA
CATGATCGACTATTTCGGACGGCCGTATGAAGGCATCGCCGTGTTCGACGGC
AAAAAGATCACTGTAACAGGGACCCTGTGGAACGGCAACAAAATTATCGAC
GAGCGCCTGATCAACCCCGACGGCTCCCTGCTGTTCCGAGTAACCATCAACG
GAGTGACCGGCTGGCGGCTGTGCGAACGCATTCTGGCGTAGACCGGTAAAAA
CAAAAAACATAGGTCAAGATATTGTTGAGTTTATTTGAGGAGGTTTATACACAGA
TGAACCACAATGCGGTGACGTATTGTTAAAAATCCTGCTTAATGCTGGAAAA
TCCCCAATCTTAGGATTTGCATACGACTTATTCTTTATAATAGTATTATTAATA
GGCGTGAAAATTGCAATGA
SEQ ID NO: 7 Construct B; Underlined: Intronic sequence; Bolded:
Nanoluciferase; Bolded Underlined: CVB3 IRES; Double Underline: homology arms;
Italic: short exons.
ACGGGGAAAATCAGCAGGGGTGAGAAGTTTACATACTTCAGAAGCCTCTCAG
AGACTACATGCAGGAGATCTTATGTCGACATAAGATCATGATATAGTCCGAT
CAATAAAGAAATTTATTGCGTATAGTAAGAGGATTTAATATTTATATTAAATC
TGTAACTATCAACATAAATGCTCTGTAAATAATGCAACGTTTTTTGTTTTTGGAT
CCTTAAAACAGCCTGTGGGTTGATCCCACCCACAGGCCCATTGGGCGCT
AGCACTCTGGTATCACGGTACCTTTGTGCGCCTGTTTTATACCCCCTCCC
CCAACTGTAACTTAGAAGTAACACACACCGATCAACAGTCAGCGTGGCA
CACCAGCCACGTTTTGATCAAGCACTTCTGTTACCCCGGACTGAGTATCA
ATAGACTGCTCACGCGGTTGAAGGAGAAAGCGTTCGTTATCCGGCCAAC
TACTTCGAAAAACCTAGTAACACCGTGGAAGTTGCAGAGTGTTTCGCTC
AGCACTACCCCAGTGTAGATCAGGTCGATGAGTCACCGCATTCCCCACG
GGCGACCGTGGCGGTGGCTGCGTTGGCGGCCTGCCCATGGGGAAACCC
ATGGGACGCTCTAATACAGACATGGTGCGAAGAGTCTATTGAGCTAGTT
GGTAGTCCTCCGGCCCCTGAATGCGGCTAATCCTAACTGCGGAGCACAC
ACCCTCAAGCCAGAGGGCAGTGTGTCGTAACGGGCAACTCTGCAGCGGA
ACCGACTACTTTGGGTGTCCGTGTTTCATTTTATTCCTATACTGGCTGCT
TATGGTGACAATTGAGAGATCGTTACCATATAGCTATTGGATTGGCCATC
CGGTGACTAATAGAGCTATTATATATCCCTTTGTTGGGTTTATACCACTT
AGCTTGAAAGAGGTTAAAACATTACAATTCATTGTTAAGTTGAATACAGC
AAAATGGACGTCGTCTTCACACTCGAAGATTTCGTTGGGGACTGGCGACA
GACAGCCGGCTACAACCTGGACCAAGTCCTTGAACAGGGAGGTGTGTCC
AGTTTGTTTCAGAATCTCGGGGTGTCCGTAACTCCGATCCAAAGGATTGT
CCTGAGCGGTGAAAATGGGCTGAAGATCGACATCCATGTCATCATCCCG
TATGAAGGTCTGAGCGGCGACCAAATGGGCCAGATCGAAAAAATTTTTA
AGGTGGTGTACCCTGTGGATGATCATCACTTTAAGGTGATCCTGCACTATG
GCACACTGGTAATCGACGGGGTTACGCCGAACATGATCGACTATTTCGGACG
GCCGTATGAAGGCATCGCCGTGTTCGACGGCAAAAAGATCACTGTAACAGGG
ACCCTGTGGAACGGCAACAAAATTATCGACGAGCGCCTGATCAACCCCGACG
GCTCCCTGCTGTTCCGAGTAACCATCAACGGAGTGACCGGCTGGCGGCTGTG
CGAACGCATTCTGGCGTAGACCGGTAAAAACAAAAAACTTGAGTTTATTTGAG
GAGGTTTATACACAGATGAACCACAATGCGGTGACGTATTGTTAAAAATCCTG
CTTAATGCTGGAAAATCCCCAATCTTAGGATTTGCATACGACTTATTCTTTAT
AATAGTATTATTAATAGGCGTGAAAATTGCAATGA
SEQ ID NO: 8 Construct C; Underlined: Intronic sequence; Bolded:
Nanoluciferase; Bolded Underlined: CVB3 IRES; Double Underline: homology arms;
Italic: short exons; Italic underlined: extended exons;
ACGGGGAAAATCAGCAGGGGTGAGAAGTTTACATACTTCAGAAGCCTCTCAG
AGACTACATGCAGGAGATCTTATGTCGACATAAGATCATGATATAGTCCGAT
CAATAAAGAAATTTATTGCGTATAGTAAGAGGATTTAATATTTATATTAAATC
TGTAACTATCAACATAAATGCTCTGTAAATAATGCAACTTTAAACAGATTCTTTGC
ATTGTTTTTTGTTTTTATCCGTTGACCTTAAACGGTCGTGTGGGTTCAAGTCCC
TCCACCCCCACGCCGGAAACGCAATAGCCGGGATCCTTAAAACAGCCTGTG
GGTTGATCCCACCCACAGGCCCATTGGGCGCTAGCACTCTGGTATCACG
GTACCTTTGTGCGCCTGTTTTATACCCCCTCCCCCAACTGTAACTTAGAA
GTAACACACACCGATCAACAGTCAGCGTGGCACACCAGCCACGTTTTGA
TCAAGCACTTCTGTTACCCCGGACTGAGTATCAATAGACTGCTCACGCG
GTTGAAGGAGAAAGCGTTCGTTATCCGGCCAACTACTTCGAAAAACCTA
GTAACACCGTGGAAGTTGCAGAGTGTTTCGCTCAGCACTACCCCAGTGT
AGATCAGGTCGATGAGTCACCGCATTCCCCACGGGCGACCGTGGCGGTG
GCTGCGTTGGCGGCCTGCCCATGGGGAAACCCATGGGACGCTCTAATAC
AGACATGGTGCGAAGAGTCTATTGAGCTAGTTGGTAGTCCTCCGGCCCC
TGAATGCGGCTAATCCTAACTGCGGAGCACACACCCTCAAGCCAGAGGG
CAGTGTGTCGTAACGGGCAACTCTGCAGCGGAACCGACTACTTTGGGTG
TCCGTGTTTCATTTTATTCCTATACTGGCTGCTTATGGTGACAATTGAGA
GATCGTTACCATATAGCTATTGGATTGGCCATCCGGTGACTAATAGAGCT
ATTATATATCCCTTTGTTGGGTTTATACCACTTAGCTTGAAAGAGGTTAA
AACATTACAATTCATTGTTAAGTTGAATACAGCAAAATGGACGTCGTCTT
CACACTCGAAGATTTCGTTGGGGACTGGCGACAGACAGCCGGCTACAAC
CTGGACCAAGTCCTTGAACAGGGAGGTGTGTCCAGTTTGTTTCAGAATC
TCGGGGTGTCCGTAACTCCGATCCAAAGGATTGTCCTGAGCGGTGAAAA
TGGGCTGAAGATCGACATCCATGTCATCATCCCGTATGAAGGTCTGAGC
GGCGACCAAATGGGCCAGATCGAAAAAATTTTTAAGGTGGTGTACCCTG
TGGATGATCATCACTTTAAGGTGATCCTGCACTATGGCACACTGGTAATCGA
CGGGGTTACGCCGAACATGATCGACTATTTCGGACGGCCGTATGAAGGCATC
GCCGTGTTCGACGGCAAAAAGATCACTGTAACAGGGACCCTGTGGAACGGCA
ACAAAATTATCGACGAGCGCCTGATCAACCCCGACGGCTCCCTGCTGTTCCG
AGTAACCATCAACGGAGTGACCGGCTGGCGGCTGTGCGAACGCATTCTGGCG
TAGACCGGTAAAAACAAAAAACATAGGTCAAGATATTGTTGAGTTTATTTGAGGA
GGTTTATACACAGATGAACCACAATGCGGTGACGTATTGTTAAAAATCCTGCT
TAATGCTGGAAAATCCCCAATCTTAGGATTTGCATACGACTTATTCTTTATAA
TAGTATTATTAATAGGCGTGAAAATTGCAATGAG
SEQ ID NO: 9 Construct D; Underlined: Intronic sequence.
ACGGGGAAAATCAGCAGGGGTGAGAAGTTTACATACTTCAGAAGCCTCTCAG
AGACTACATGCAGGAGATCTTATGTCGACATAAGATCATGATATAGTCCGAT
CAATAAAGAAATTTATTGCGTATAGTAAGAGGATTTAATATTTATATTAAATC
TGTAACTATCAACATAAATGCTCTGTAAATAATGCAACTTGAGTTTATTTGAG
GAGGTTTATACACAGATGAACCACAATGCGGTGACGTATTGTTAAAAATCCT
GCTTAATGCTGGAAAATCCCCAATCTTAGGATTTGCATACGACTTATTCTTTA
TAATAGTATTATTAATAGGCGTGAAAATTGCAATGAG
SEQ ID NO: 10 Construct E; Underlined: Intronic sequence; Bolded:
Nanoluciferase; Bolded Underlined: CVB3 IRES; Italic: short exons; Italic underlined:
extended exons.
ACGGGGAAAATCAGCAGGGGTGAGAAGTTTACATACTTCAGAAGCCTCTCAG
AGACTACATGCAGGAGATCTTATGTCGACATAAGATCATGATATAGTCCGAT
CAATAAAGAAATTTATTGCGTATAGTAAGAGGATTTAATATTTATATTAAATC
TGTAACTATCAACATAAATGCTCTGTAAATAATGCAACTTTAAACAGATTCTTTGC
ATTGGATCCTTAAAACAGCCTGTGGGTTGATCCCACCCACAGGCCCATTG
GGCGCTAGCACTCTGGTATCACGGTACCTTTGTGCGCCTGTTTTATACCC
CCTCCCCCAACTGTAACTTAGAAGTAACACACACCGATCAACAGTCAGC
GTGGCACACCAGCCACGTTTTGATCAAGCACTTCTGTTACCCCGGACTG
AGTATCAATAGACTGCTCACGCGGTTGAAGGAGAAAGCGTTCGTTATCC
GGCCAACTACTTCGAAAAACCTAGTAACACCGTGGAAGTTGCAGAGTGT
TTCGCTCAGCACTACCCCAGTGTAGATCAGGTCGATGAGTCACCGCATT
CCCCACGGGCGACCGTGGCGGTGGCTGCGTTGGCGGCCTGCCCATGGG
GAAACCCATGGGACGCTCTAATACAGACATGGTGCGAAGAGTCTATTGA
GCTAGTTGGTAGTCCTCCGGCCCCTGAATGCGGCTAATCCTAACTGCGG
AGCACACACCCTCAAGCCAGAGGGCAGTGTGTCGTAACGGGCAACTCTG
CAGCGGAACCGACTACTTTGGGTGTCCGTGTTTCATTTTATTCCTATACT
GGCTGCTTATGGTGACAATTGAGAGATCGTTACCATATAGCTATTGGATT
GGCCATCCGGTGACTAATAGAGCTATTATATATCCCTTTGTTGGGTTTAT
ACCACTTAGCTTGAAAGAGGTTAAAACATTACAATTCATTGTTAAGTTGA
ATACAGCAAAATGGACGTCGTCTTCACACTCGAAGATTTCGTTGGGGACT
GGCGACAGACAGCCGGCTACAACCTGGACCAAGTCCTTGAACAGGGAG
GTGTGTCCAGTTTGTTTCAGAATCTCGGGGTGTCCGTAACTCCGATCCAA
AGGATTGTCCTGAGCGGTGAAAATGGGCTGAAGATCGACATCCATGTCA
TCATCCCGTATGAAGGTCTGAGCGGCGACCAAATGGGCCAGATCGAAAA
AATTTTTAAGGTGGTGTACCCTGTGGATGATCATCACTTTAAGGTGATCCT
GCACTATGGCACACTGGTAATCGACGGGGTTACGCCGAACATGATCGACTAT
TTCGGACGGCCGTATGAAGGCATCGCCGTGTTCGACGGCAAAAAGATCACTG
TAACAGGGACCCTGTGGAACGGCAACAAAATTATCGACGAGCGCCTGATCAA
CCCCGACGGCTCCCTGCTGTTCCGAGTAACCATCAACGGAGTGACCGGCTGG
CGGCTGTGCGAACGCATTCTGGCGTAAACCGGTATAGGTCAAGATATTGTTGAG
TTTATTTGAGGAGGTTTATACACAGATGAACCACAATGCGGTGACGTATTGTTA
AAAATCCTGCTTAATGCTGGAAAATCCCCAATCTTAGGATTTGCATACGACTT
ATTCTTTATAATAGTATTATTAATAGGCGTGAAAATTGCAATGAG
SEQ ID NO: 11 Construct F; Underlined: Intronic sequence.
GGCGTGAAAATTGCAATGACACGGGGAAAATCAGCAGGGGTGAGAAGTTTA
CATACTTCAGAAGCCTCTCAGAGACTACATGCAGGAGATCTTATGTCGACAT
AAGATCATGATATAGTCCGATCAATAAAGAAATTTATTGCGTATAGTAAGAG
GATTTAATATTTATATTAAATCTGTAACTATCAACATAAATGCTCTGTAAATA
ATGCAACTTTAAACAGATTCTTTGCATTACATTTCTTATTACCTTTTGTATTAG
CTGCTTTAGCATTAATGCATTTAATAGCTATGCATGATACAGTAGGATCAGGT
AATCCTTTAGGTATTTCTGCTAATTACGATAGATTACCTTTTGCTCCTTATTTT
ATATACCGGTGGAATTCGACGTCAACAGACAACATACAATACGGCTATGGAT
CCGAGGTTTATATTATGGATCTTACAAAACACCTAGAACTTTAACATGAGCTA
TTGGAACAGTAATACTAATAGTTATGATGGCCACAGCCTTCTTAGGTTATGTT
TTACCTTATGGTCAAATGAGTTTATGAGGTGCTACAGTTATTACTAACCTAAT
GAGTGCTATACCTTGAATAGGTCAAGATATTGTTGAGTTTATTTGAGGAGGTT
TATACACAGATGAACCACAATGCGGTGACGTATTGTTAAAAATCCTGCTTAA
TGCTGGAAAATCCCCAATCTTAGGATTTGCATACGACTTATTCTTTATAATAG
TATTATTAATAG
SEQ ID NO: 12 Construct G; Underlined: Intronic sequence.
TCCTGCTTAATGCTGGAAAATCCCCAATCTTAGGATTTGCATACGACTTATTC
TTTATAATAGTATTATTAATAGGCGTGAAAATTGCAATGACACGGGGAAAAT
CAGCAGGGGTGAGAAGTTTACATACTTCAGAAGCCTCTCAGAGACTACATGC
AGGAGATCTTATGTCGACATAAGATCATGATATAGTCCGATCAATAAAGAAA
TTTATTGCGTATAGTAAGAGGATTTAATATTTATATTAAATCTGTAACTATCA
ACATAAATGCTCTGTAAATAATGCAACTTTAAACAGATTCTTTGCATTACATT
TCTTATTACCTTTTGTATTAGCTGCTTTAGCATTAATGCATTTAATAGCTATGC
ATGATACAGTAGGATCAGGTAATCCTTTAGGTATTTCTGCTAATTACGATAGA
TTACCTTTTGCTCCTTATTTTATATACCGGTGGAATTCGACGTCAACAGACAA
CATACAATACGGCTATGGATCCGAGGTTTATATTATGGATCTTACAAAACAC
CTAGAACTTTAACATGAGCTATTGGAACAGTAATACTAATAGTTATGATGGC
CACAGCCTTCTTAGGTTATGTTTTACCTTATGGTCAAATGAGTTTATGAGGTG
CTACAGTTATTACTAACCTAATGAGTGCTATACCTTGAATAGGTCAAGATATT
GTTGAGTTTATTTGAGGAGGTTTATACACAGATGAACCACAATGCGGTGACG
TATTGTTAAAAAGAAAAGAAGAGCActc
SEQ ID NO: 13 Construct H; Underlined: Intronic sequence.
ACATGCAGGAGATCTTATGTCGACATAAGATCATGATATAGTCCGATCAATA
AAGAAATTTATTGCGTATAGTAAGAGGATTTAATATTTATATTAAATCTGTAA
CTATCAACATAAATGCTCTGTAAATAATGCAACTTTAAACAGATTCTTTGCAT
TACATTTCTTATTACCTTTTGTATTAGCTGCTTTAGCATTAATGCATTTAATAG
CTATGCATGATACAGTAGGATCAGGTAATCCTTTAGGTATTTCTGCTAATTAC
GATAGATTACCTTTTGCTCCTTATTTTATATACCGGTGGAATTCGACGTCAAC
AGACAACATACAATACGGCTATGGATCCGAGGTTTATATTATGGATCTTACA
AAACACCTAGAACTTTAACATGAGCTATTGGAACAGTAATACTAATAGTTAT
GATGGCCACAGCCTTCTTAGGTTATGTTTTACCTTATGGTCAAATGAGTTTAT
GAGGTGCTACAGTTATTACTAACCTAATGAGTGCTATACCTTGAATAGGTCA
AGATATTGTTGAGTTTATTTGAGGAGGTTTATACACAGATGAACCACAATGC
GGTGACGTATTGTTAAAAATCCTGCTTAATGCTGGAAAATCCCCAATCTTAGG
ATTTGCATACGACTTATTCTTTATAATAGTATTATTAATAGGCGTGAAAATTG
CAATGACACGGGGAAAATCAGCAGGGGTGAGAAGTTTACATACTTCAGAAG
CCTCTCAGAGACTGAAAAGAAGAGCActc
SEQ ID NO: 14 Little intron; Underlined: Intronic sequence.
GAGGTTTATATTATGGATCTTACAAAACACCTAGAACTTTAACATGAGCTATT
GGAACAGTAATACTAATAGTTATGATGGCCACAGCCTTCTTAGGTTATGTTTT
ACCTTATGGTCAAATGAGTTTATGAGGTGCTACAGTTATTACTAACCTAATGA
GTGCTATACCTTGAATAGGTCAAGATATTGTTGAGTTTATTTGAGGAGGTTTA
TACACAGATGAACCACAATGCGGTGACGTATTGTTAAAAATCCTGCTTAATG
CTGGAAAATCCCCAATCTTAGGATTTGCATACGACTTATTCTTTATAATAGTA
TTATTAATAGGCGTGAAAATTGCAATGACACGGGGAAAATCAGCAGGGGTG
AGAAGTTTACATACTTCAGAAGCCTCTCAGAGACTACATGCAGGAGATCTTA
TGTCGACATAAGATCATGATATAGTCCGATCAATAAAGAAATTTATTGCGTA
TAGTAAGAGGATTTAATATTTATATTAAATCTGTAACTATCAACATAAATGCT
CTGTAAATAATGCAACTTTAAACAGATTCTTTGCATTACATTTCTTATTACCTT
TTGTATTAGCTGCTTTAGCATTAATGCATTTAATAGCTATGCATGATACAGTA
GGATCAGGTAATCCTTTAGGTATTTCTGCTAATTACGATAGATTACCTTTTGC
TCCTTATTTTATATACCGGT

What is claimed is:

1. A method of making circular RNA comprising:
a) transcribing a vector to form a precursor RNA, wherein the vector comprises operably linked elements ordered as follows and excludes a complementarity sequence:
i. a 3′ Group I intron or 3′ Group II intron sequence containing a 3′ splice site dinucleotide,
ii. a first non-coding sequence, and
iii. a corresponding 5′ Group I intron or 5′ Group II intron sequence containing a 5′ splice site dinucleotide; and
b) contacting the precursor RNA with a paired Group I or Group II maturase polypeptide to allow formation of the circular RNA.

2. The method of claim 1, wherein the vector further comprises an internal ribosome entry site (IRES), a protein coding sequence and a second non-coding sequence, in that order, between the elements of a) ii. and a) iii.

3. A method of making circular RNA comprising:
a) transcribing a vector to form a precursor RNA, wherein the vector comprises operably linked elements ordered as follows:
i. a 3′ Group I intron or 3′ Group II intron sequence containing a 3′ splice site dinucleotide,
ii. a first non-coding sequence,
iii. a 5′ complementarity sequence,
iv. a 3′ complementarity sequence;
v. a corresponding 5′ Group I intron or 5′ Group II intron sequence containing a 5′ splice site dinucleotide, and
vi. and
b) contacting the precursor RNA with a paired Group I or Group II maturase polypeptide to allow formation of the circular RNA.

4. The method of claim 3, wherein the vector further comprises an internal ribosome entry site (IRES), a protein coding sequence and a second non-coding sequence, in that order, between the elements of a) iii. and a) iv.

5. The method of any one of claims 1-4, wherein the maturase polypeptide and the precursor RNA are contacted under conditions comprising a temperature of between 20° C. and 45° C. and a magnesium concentration of between 100 micromolar and 25 millimolar.

6. The method of claim 2 or claim 4, wherein the vector further comprises a second IRES, a second protein coding sequence, and a third non-coding sequence between the second non-coding sequence and the 5′ Group II intron sequence containing a 5′ splice site dinucleotide.

7. The method of claim 6, wherein the vector further comprises a third IRES, a third protein coding sequence, and a fourth non-coding sequence between the third non-coding sequence and the 5′ Group II intron sequence containing a 5′ splice site dinucleotide.

8. The method of any one of claims 1-7, wherein the Group I intron is an Aspergillus nidulans COB1 intron.

9. The method of claim 8, wherein the paired Group I maturase is an Aspergillus nidulans I-AniI maturase.

10. The method of claim 9, wherein the Aspergillus nidulans I-AniI maturase has a reduced DNA endonuclease activity and/or and increased splicing activity.

11. The method of claim 9 or claim 10, wherein the Aspergillus nidulans I-AniI maturase is N-terminally truncated.

12. The method of any one of claims 1-7, wherein the Group II intron is selected from an Oryza sativa trnK gene, tRNAs (V-UAC, I-GAU, A-UGC, and K-UUU), ribosomal proteins (rp12 and rps12), and chloroplast ATPase (atpF).

13. The method of claim 12, wherein the paired Group II maturase is an Oryza sativa maturase K.

14. The method of any one of claims 1-7, wherein the Group II intron is a Lactococcus lactis LtrA intron.

15. The method of claim 14, wherein the paired Group II maturase is a Lactococcus lactis LtrA maturase.

16. The method of any one of claims 1-7, wherein the Group II intron is a Lactococcus lactis LtrB intron.

17. The method of claim 16, wherein the paired Group II maturase is a Lactococcus lactis LtrB maturase.

18. The method of any one of claims 1-7, wherein the Group II intron is a IIA intron a12 and the paired Group II maturase is cyt-19 or Mss116p.

19. The method of any one of claims 1-7, wherein the Group II intron is aI5γ and bI1 and the paired Group II maturase is cyt-19.

20. The method of any one of claims 1-7, wherein the Group II intron is nad1 i4 and the Group IL paired intron is MatR.

21. The method of any one of claims 1-20, wherein one or more of the non-coding sequences are between 10 and 40 nucleotides.

22. The method of any one of claims 1-20, wherein one or more of the non-coding sequences are between 15 and 50 nucleotides.

23. The method of any one of claims 1-20, wherein one or more of the non-coding sequences are between 18 and 22 nucleotides.

24. The method of any one of claims 1-23, wherein one or more of the non-coding sequences comprise a poly(A) sequence.

25. The method of any one of claims 1-24, wherein one or more of the non-coding sequences comprise only A and C nucleotides.

26. The method of any one of claims 1-25, wherein the IRES has a sequence of an aptamer to eIF4G, a Homo sapiens cDNA FLJ43058, an Acute bee paralysis virus IRES, an Aphid lethal paralysis virus IRES, an Avian encephalomyelitis virus IRES, a Bovine viral diarrhea virus 1 IRES, a Canine Scamper IRES, a Classical swine fever virus IRES, a Cosavirus, a Coxsackievirus A (CVB1/2) IRES, a Cricket paralysis virus IRES, a Crucifer tobamo virus IRES, a Drosophila antennapedia IRES, a Diresapivirus B1 IRES, a Drosophila C Virus IRES, a Drosophila hairless IRES, a Drosophila reaper IRES, a Drosophila Ubx IRES, a Ectropis obliqua picoma-like virus IRES, a Encephalomyocarditis virus (EMCV) IRES, an Equine rhinitis virus IRES, a foot and mouth disease virus IRES, a Guanxi changeable lizard picomavirus 2 IRES, a Hepatitis A virus IRES, a Hepatitis C virus IRES, a Hepatitis GB virus IRES, a Hibiscus chlorotic ringspot virus IRES, a Himetobi P virus IRES, a Homalodisca coagulata virus-1 IRES, a Human AML1/RUNX1 gene IRES, a Human AQP4 gene IRES, a Human ATIR gene IRES, a Human BAG-1 gene IRES, a Human BCL2 gene IRES, a Human BiP gene IRES, a Human c-IAP1 gene IRES, a Human c-myc gene IRES, a Human c-src gene IRES, a Human eIF4G gene IRES, a Human enterovirus 71 IRES, a Human FGF-1 gene IRES, a Human FGF2 gene IRES, a Human Immunodeficiency Virus type 1 IRES, a Human LEF1 gene IRES, a Human n.myc gene IRES, a Human p27kip1 gene IRES, a Human p53 gene IRES, a Human papillomavirus IRES, a Human PDGF2/c-sis gene IRES, a Human Pim-1 gene IRES, a Human rhinovirus 2 IRES, a Human SFTPA1 gene IRES, a Human UNR gene IRES, a Human VEGF-A gene IRES, a Human XIAP gene IRES, a Kashmir bee virus IRES, a Mouse eukaryotic translation initiation factor 1A domain containing 14 (Eiflad14) gene IRES, a Mouse Gtx gene IRES, a Mouse HIF1 alpha gene IRES, a Mouse NDST4L gene IRES, a Mouse Rbm3 gene IRES, a Mouse UtrA gene IRES, a Parechovirus IRES, a Pink-eared duck picomavirus IRES, a Plautia stali intestine virus IRES, a poliovirus 1 IRES, a Reticuloendotheliosis virus IRES, a Rhopalosiphum padi virus IRES, a Rous sarcoma virus IRES, a S. cerevisiae TFIID gene IRES. a S. cerevisiae YAP1 gene IRES, a Salivirus IRES, a Simian picomavirus IRES, a simian Virus 40 IRES, a Solenopsis invicta virus 1 IRES, a Taura syndrome virus IRES, a Theiler's encephalomyelitis virus IRES, or a Triatoma virus IRES.

27. The method of any one of claims 4-26, wherein the protein coding sequence encodes a viral protein, a eukaryotic protein, or a prokaryotic protein.

28. The method of any one of claims 4-26, wherein the protein coding sequence encodes a human protein.

29. The method of any one of claims 4-26, wherein the protein coding sequence encodes an antibody.

30. The method of claim 29, wherein the antibody is bispecific.

31. The method of any one of claims 4-26, wherein the protein coding sequence encodes a viral antigen or a bacterial antigen.

32. A circular RNA made according to the method of any one of claims 1-31.

33. The circular RNA of claim 32, wherein the RNA comprises between 300 and 12000 nucleotides.

34. A pharmaceutical composition comprising the circular RNA of claim 29 or 30 and a pharmaceutically acceptable nanocarrier selected from the group consisting of a lipid nanoparticle, a lipid, a lipid polymer, a lipo-polymeric hybrid, an exosome and a leukosome.

35. A method of isolating circular RNA comprising:
a) obtaining a mixture of linear RNA and circular RNA created by the method of any one of claims 1-31:
b) contacting the mixture with one or more fixed 5′ binding proteins and one or more fixed 3′ binding proteins; and
c) isolating the circular RNA.

36. The method of claim 35, wherein the 5′ binding protein is a Saccharomyces cerevisiae DXO 5′ binding protein, or a homolog thereof.

37. The method of claim 35 or claim 36, wherein the 3′ binding protein is a nuclease inactivated RNAse R.

38. A method of isolating circular RNA comprising:
a) obtaining a mixture of linear RNA and circular RNA created by the method of any one of claims 1-31; and
b) isolating the circular RNA using size exclusion chromatography.


SEQUENCE LISTING (51 sequences)

SEQ ID NO: 1 - DNA, 1629 bp, Oryza sativa
  misc_feature 1..1629: note = Description of sequence:
  source 1..1629: mol_type = genomic DNA
atgcaacaccctgttctgaccatattgtactatgtatcaccatttgataaaccgagaaatatttctctctctctgattcaagtagaaatacaaatggaaaaattcgagggttattcagaaaaacttaaatttcctagacaatatttcgtctacccactactctttcaggagtacatttatgtgtttgcccatgattatggattaaatggttccgaacttgtcgaaattattggttctaataacaagaaatttagttcactacttgtgaaacggttaatgattcgaatgtatcagcagaatttttggattaatttggttaatcatcctaaccaagatcgattattggattacaacaattttttttattctgagttttattctcagattctatctgaggggtttgcgatcgttgtagaaatcccattctcgctacgagaacagtcttgtccggaagaaaaagaaataccaaagtttcagaatttacgatctattcattcaatatttccttttttagaagacaaatttttgcacttacattatctagcacatatagaaataccctatcctatccatttggatatcttgcttcaactccttcaataccggatccaagatgttccatccttgcatttattgcgattctttctcaactactactcgaattggaatagttttattacttcaatgaaatccatttttattttgaaaaaagaaaataaaagactatttcggttcctatataactcttatgtatcagaatatgaatttttcttgttgtttcttcgtaaacaatcttcttgcttgcgattaacttcttccggaacctttctggaacgaatcatcttttctaggaagatggaacattttgggttaatgtaccctgctttttttcggaaaaccatatggttcgttatggatccccttatgcattatgttcgatatcaaggaaaggcaattcttgcatcaaaaggaactcttcttttgaagaagaaatggaaatgttaccttgtccgtttgtggcaatattctttctctttttggactcaaccgcaaaggatccatctaaaccaattagaaaactcttgcttcgattttctggggtacttttcaagtgtaccaataaattctttgttagtaaggaatcaaatgctggagaattcatttctaatagatactcaaatgaaaaaattcgataccaaagtccctgttactcctctcattggatctttagcaaaagcccaattttgtactggatcggggcatcctattagtaaaccaatttggaccgatttatcggattgggatattcttgatcggtttggtcggatatgtagaaatctttttcattatcacagtggatcttcaaaaaagaagactttgtatcgactaaagtatatacttcgactttcatgcgctagaactttagctcgtaaacataaaagcacggtacgagcttttatgcaatggttgggttcggtatttttagaagaattttttacggaagaagagcaagttttttctttgatgttcgccaaaacaacttacttttctttccgtggatcgcacagtgagcgtatttggtatttggatattctccgtatcaatgatctggtgaatcctcttaattaa

SEQ ID NO: 2 - protein, 626 aa, Neurospora crassa
  REGION 1..626: note = Description of sequence:
MRTSVLRQAGLCRAALAARHLQISSKPSAALLSQVTRAIAVQSLPSASLPRFYSAEATAQSNTAASNGLVTRFADLAALGVHENVVRAITHGMGYENMTEVQSMTISPALKGKDIVAQAKTGTGKTLGFLVPVIQKIITQDPDLAHRFGGKRARSDDIRAIIISPTRELAEQIGEEARKLVKGTGIIVQTAVGGTQKNAMLYKTRQQGCHILVGTPGRLNDLLSDSHSGIDAPRLSTLVLDEADRMLEVGFNEELRQIINYLPDRKVLPRQTLLYSATIPKDVVGLARSYIDKNNFEFVQTVKADEVLTHDRIPQYIVPCKGFENIYPAMLELIEKALNESRTNPEALPFKAIVFLPTTAEVIMANAIFKRLQWKFKHIPKTWDIHSKLTQNARTRAADEFKNARTGILFSSDVTARGMDFPNVSHVIQTHIPPNREQYIHRLGRTGRANKPGQGWLIVPDIELHAARSRLPGLPIKRNDELECASVNAADSGADKHANFQHILDAASRLPEDLFKDCYSSYLGGALQGIDRQALVYALNDLAKFGWGLEEPPAVRQSIMKHMGRVQGLRVETREHSMRPMGSGPGHRRDFNSRGPRRQSDDPFENALHRAQDLDRRPTRRQQASF

SEQ ID NO: 3 - protein, 276 aa, Aspergillus nidulans
  REGION 1..276: note = Description of sequence:
MTRGKSAGVRSLHTSEASQRLHAGDLTYAYLVGLFEGDGYFSITKKGKYLTYELGIELSIKDVQLIYKIKKILGIGIVSFRKRNEIEMVALRIRDKNHLKSFILPIFEKYPMFSNKQYDYLRFRNALLSGIISLEDLPDYTRSDEPLNSIESIINTSYFSAWLVGFIEAEGCFSVYKLNKDDDYLIASFDIAQRDGDILISAIRKYLSFTTKVYLDKTNCSKLKVTSVRSVENIIKFLQNAPVKLLGNKKLQYLLWLKQLRKISRYSEKIKIPSNY

SEQ ID NO: 4 - protein, 115 aa, Aspergillus nidulans
  REGION 1..115: note = Description of sequence:
WLVGFIEAEGCFSVYKLNKDDDYLIASFDIAQRDGDILISAIRKYLSFTTKVYLDKTNCSKLKVTSVRSVENIIKFLQNAPVKLLGNKKLQYLLWLKQLRKISRYSEKIKIPSNY

SEQ ID NO: 5 - protein, 442 aa, Saccharomyces cerevisiae
  REGION 1..442: note = Description of sequence:
MSTEQDAVLGLAKDLEGINLLTVPNLERGHQSKLCKEKTTSDSSSSRKPSQQRDNYRKRRPKLICIPYTSFLHTGMHNFLTKPPRDIFHESKEVALFTNGRAYTILRKDLIPNLKESIAELYESSLLEAKKRKVPYLGHDLFANIDEFVPMTISELDSVSPCFSYIENWILDNPGKDFKIGKKFTVVTTRHHIVDLTMHLFNRRNRQTSLIVTYMGAGLLSFCRNVKKDSQMSKEGIYSNDPNMKKICYSGFEFENWVTENSKVADLTGSKCPIFSLVESKLSEEIGLLIRCEMDAFNPVSETNTELKCFAPLSMHNSNHRRKLLKTWVQTGLLPNSDIMIGLRDSHSGQLLDIQWYSRDLLCKKFNHPGLPTNKKELNYNAQIAVEWCHYCIEAICKLVEANISDYSSTKPESFEIGIDTNNAIVITKLKTTPRNVELFGM

SEQ ID NO: 6 - DNA, 1689 bp, synthetic construct
  misc_feature 1..1689: note = Description of sequence: circular nucleic acid component
  source 1..1689: mol_type = other DNA
acggggaaaatcagcaggggtgagaagtttacatacttcagaagcctctcagagactacatgcaggagatcttatgtcgacataagatcatgatatagtccgatcaataaagaaatttattgcgtatagtaagaggatttaatatttatattaaatctgtaactatcaacataaatgctctgtaaataatgcaactttaaacagattctttgcattgttttttgtttttggatccttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggacgtcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtagaccggtaaaaacaaaaaacataggtcaagatattgttgagtttatttgaggaggtttatacacagatgaaccacaatgcggtgacgtattgttaaaaatcctgcttaatgctggaaaatccccaatcttaggatttgcatacgacttattctttataatagtattattaataggcgtgaaaattgcaatga

SEQ ID NO: 7 - DNA, 1652 bp, synthetic construct
  misc_feature 1..1652: note = Description of sequence: circular nucleic acid component
  source 1..1652: mol_type = other DNA
acggggaaaatcagcaggggtgagaagtttacatacttcagaagcctctcagagactacatgcaggagatcttatgtcgacataagatcatgatatagtccgatcaataaagaaatttattgcgtatagtaagaggatttaatatttatattaaatctgtaactatcaacataaatgctctgtaaataatgcaacgttttttgtttttggatccttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggacgtcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtagaccggtaaaaacaaaaaacttgagtttatttgaggaggtttatacacagatgaaccacaatgcggtgacgtattgttaaaaatcctgcttaatgctggaaaatccccaatcttaggatttgcatacgacttattctttataatagtattattaataggcgtgaaaattgcaatga

SEQ ID NO: 8 - DNA, 1758 bp, synthetic construct
  misc_feature 1..1758: note = Description of sequence: circular nucleic acid component
  source 1..1758: mol_type = other DNA
acggggaaaatcagcaggggtgagaagtttacatacttcagaagcctctcagagactacatgcaggagatcttatgtcgacataagatcatgatatagtccgatcaataaagaaatttattgcgtatagtaagaggatttaatatttatattaaatctgtaactatcaacataaatgctctgtaaataatgcaactttaaacagattctttgcattgttttttgtttttatccgttgaccttaaacggtcgtgtgggttcaagtccctccacccccacgccggaaacgcaatagccgggatccttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggacgtcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtagaccggtaaaaacaaaaaacataggtcaagatattgttgagtttatttgaggaggtttatacacagatgaaccacaatgcggtgacgtattgttaaaaatcctgcttaatgctggaaaatccccaatcttaggatttgcatacgacttattctttataatagtattattaataggcgtgaaaattgcaatgag

SEQ ID NO: 9 - DNA, 352 bp, synthetic construct
  misc_feature 1..352: note = Description of sequence: circular nucleic acid component
  source 1..352: mol_type = other DNA
acggggaaaatcagcaggggtgagaagtttacatacttcagaagcctctcagagactacatgcaggagatcttatgtcgacataagatcatgatatagtccgatcaataaagaaatttattgcgtatagtaagaggatttaatatttatattaaatctgtaactatcaacataaatgctctgtaaataatgcaacttgagtttatttgaggaggtttatacacagatgaaccacaatgcggtgacgtattgttaaaaatcctgcttaatgctggaaaatccccaatcttaggatttgcatacgacttattctttataatagtattattaataggcgtgaaaattgcaatgag

SEQ ID NO: 10 - DNA, 1664 bp, synthetic construct
  misc_feature 1..1664: note = Description of sequence: circular nucleic acid component
  source 1..1664: mol_type = other DNA
acggggaaaatcagcaggggtgagaagtttacatacttcagaagcctctcagagactacatgcaggagatcttatgtcgacataagatcatgatatagtccgatcaataaagaaatttattgcgtatagtaagaggatttaatatttatattaaatctgtaactatcaacataaatgctctgtaaataatgcaactttaaacagattctttgcattggatccttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggacgtcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaaccggtataggtcaagatattgttgagtttatttgaggaggtttatacacagatgaaccacaatgcggtgacgtattgttaaaaatcctgcttaatgctggaaaatccccaatcttaggatttgcatacgacttattctttataatagtattattaataggcgtgaaaattgcaatgag

SEQ ID NO: 11 - DNA, 750 bp, synthetic construct
  misc_feature 1..750: note = Description of sequence: circular nucleic acid component
  source 1..750: mol_type = other DNA
ggcgtgaaaattgcaatgacacggggaaaatcagcaggggtgagaagtttacatacttcagaagcctctcagagactacatgcaggagatcttatgtcgacataagatcatgatatagtccgatcaataaagaaatttattgcgtatagtaagaggatttaatatttatattaaatctgtaactatcaacataaatgctctgtaaataatgcaactttaaacagattctttgcattacatttcttattaccttttgtattagctgctttagcattaatgcatttaatagctatgcatgatacagtaggatcaggtaatcctttaggtatttctgctaattacgatagattaccttttgctccttattttatataccggtggaattcgacgtcaacagacaacatacaatacggctatggatccgaggtttatattatggatcttacaaaacacctagaactttaacatgagctattggaacagtaatactaatagttatgatggccacagccttcttaggttatgttttaccttatggtcaaatgagtttatgaggtgctacagttattactaacctaatgagtgctataccttgaataggtcaagatattgttgagtttatttgaggaggtttatacacagatgaaccacaatgcggtgacgtattgttaaaaatcctgcttaatgctggaaaatccccaatcttaggatttgcatacgacttattctttataatagtattattaatag

SEQ ID NO: 12 - DNA, 765 bp, synthetic construct
  misc_feature 1..765: note = Description of sequence: circular nucleic acid component
  source 1..765: mol_type = other DNA
tcctgcttaatgctggaaaatccccaatcttaggatttgcatacgacttattctttataatagtattattaataggcgtgaaaattgcaatgacacggggaaaatcagcaggggtgagaagtttacatacttcagaagcctctcagagactacatgcaggagatcttatgtcgacataagatcatgatatagtccgatcaataaagaaatttattgcgtatagtaagaggatttaatatttatattaaatctgtaactatcaacataaatgctctgtaaataatgcaactttaaacagattctttgcattacatttcttattaccttttgtattagctgctttagcattaatgcatttaatagctatgcatgatacagtaggatcaggtaatcctttaggtatttctgctaattacgatagattaccttttgctccttattttatataccggtggaattcgacgtcaacagacaacatacaatacggctatggatccgaggtttatattatggatcttacaaaacacctagaactttaacatgagctattggaacagtaatactaatagttatgatggccacagccttcttaggttatgttttaccttatggtcaaatgagtttatgaggtgctacagttattactaacctaatgagtgctataccttgaataggtcaagatattgttgagtttatttgaggaggtttatacacagatgaaccacaatgcggtgacgtattgttaaaaagaaaagaagagcactc

SEQ ID NO: 13 - DNA, 765 bp, synthetic construct
  misc_feature 1..765: note = Description of sequence: circular nucleic acid component
  source 1..765: mol_type = other DNA
acatgcaggagatcttatgtcgacataagatcatgatatagtccgatcaataaagaaatttattgcgtatagtaagaggatttaatatttatattaaatctgtaactatcaacataaatgctctgtaaataatgcaactttaaacagattctttgcattacatttcttattaccttttgtattagctgctttagcattaatgcatttaatagctatgcatgatacagtaggatcaggtaatcctttaggtatttctgctaattacgatagattaccttttgctccttattttatataccggtggaattcgacgtcaacagacaacatacaatacggctatggatccgaggtttatattatggatcttacaaaacacctagaactttaacatgagctattggaacagtaatactaatagttatgatggccacagccttcttaggttatgttttaccttatggtcaaatgagtttatgaggtgctacagttattactaacctaatgagtgctataccttgaataggtcaagatattgttgagtttatttgaggaggtttatacacagatgaaccacaatgcggtgacgtattgttaaaaatcctgcttaatgctggaaaatccccaatcttaggatttgcatacgacttattctttataatagtattattaataggcgtgaaaattgcaatgacacggggaaaatcagcaggggtgagaagtttacatacttcagaagcctctcagagactgaaaagaagagcactc

SEQ ID NO: 14 - DNA, 705 bp, synthetic construct
  misc_feature 1..705: note = Description of sequence: circular nucleic acid component
  source 1..705: mol_type = other DNA
gaggtttatattatggatcttacaaaacacctagaactttaacatgagctattggaacagtaatactaatagttatgatggccacagccttcttaggttatgttttaccttatggtcaaatgagtttatgaggtgctacagttattactaacctaatgagtgctataccttgaataggtcaagatattgttgagtttatttgaggaggtttatacacagatgaaccacaatgcggtgacgtattgttaaaaatcctgcttaatgctggaaaatccccaatcttaggatttgcatacgacttattctttataatagtattattaataggcgtgaaaattgcaatgacacggggaaaatcagcaggggtgagaagtttacatacttcagaagcctctcagagactacatgcaggagatcttatgtcgacataagatcatgatatagtccgatcaataaagaaatttattgcgtatagtaagaggatttaatatttatattaaatctgtaactatcaacataaatgctctgtaaataatgcaactttaaacagattctttgcattacatttcttattaccttttgtattagctgctttagcattaatgcatttaatagctatgcatgatacagtaggatcaggtaatcctttaggtatttctgctaattacgatagattaccttttgctccttattttatataccggt

SEQ ID NO: 15 - protein, 599 aa, Lactococcus lactis
  REGION 1..599: note = Description of sequence:
MKPTMAILERISKNSQENIDEVFTRLYRYLLRPDIYYVAYQNLYSNKGASTKGILDDTADGFSEEKIKKIIQSLKDGTYYPQPVRRMYIAKKNSKKMRPLGIPTFTDKLIQEAVRIILESIYEPVFEDVSHGFRPQRSCHTALKTIKREFGGARWFVEGDIKGCFDNIDHVTLIGLINLKIKDMKMSQLIYKFLKAGYLENWQYHKTYSGTPQGGILSPLLANIYLHELDKFVLQLKMKFDRESPERITPEYRELHNEIKRISHRLKKLEGEEKAKVLLEYQEKRKRLPTLPCTSQTNKVLKYVRYADDFIISVKGSKEDCQWIKEQLKLFIHNKLKMELSEEKTLITHSSQPARFLGYDIRVRRSGTIKRSGKVKKRTLNGSVELLIPLQDKIRQFIFDKKIAIQKKDSSWFPVHRKYLIRSTDLEIITIYNSELRGICNYYGLASNFNQLNYFAYLMEYSCLKTIASKHKGTLSKTISMFKDGSGSWGIPYEIKQGKQRRYFANFSECKSPYQFTDEISQAPVLYGYARNTLENRLKAKCCELCGTSDENTSYEIHHVNKVKNLKGKEKWEMAMIAKQRKTLVVCFHCHRHVIHKHK

SEQ ID NO: 16 - protein, 665 aa, Vicia faba
  REGION 1..665: note = Description of sequence:
MVLESIYDPEFPDTSHFRSGRGRYSALRRIKEEWGTSRWFFEFDIRKCFHTIDRHRLIPIFKEEIDDPKFFYSIHKVFSAGRLVGGEKGPYSVPHSVLLSALPGNIYLHKLDQEIGRIRQKYEIPRIRSVLLRTGRIDDQENSGEEASLNAPQDNRAIIVGRVKSIQRKAAFHSLVSSWHTPPTSTPRRRGDQKTPFVFPPSSALAAFLNKPSSLLCAAFFIEAAGAGLTPKTEFYGRECCFNNNLAMRDLIKYCKRKGLLIELGGEARLVIRSERGLARKLAPFKIKTHYFIRICYVRYADDLLLGIVGAVELLIEIQKRIAHFLQSGLNLWVGSAGSTTIAARSTVEFPGMVIREVPPRTTPIQLLRELEKRLRVKHRIHITVCHLRSAIHSKFRNLGNSIPIKQLTKGMSERGSLLDGVPLAETLGTAGVRSPQVSVLWGTVQHIWQGSRGISLLHSSGRSNAPSDVQEAVARSGMSVRKLSLYTPAGRKAAGEGGGHRAGSISSEFPIQIEAPIKKILRRLRDRGIISRRRPWPIHVACLTNVSDGDIVNWSAGIAISPLSYYRCCDNLYQVRTIVDHQIRWSAIFTLAHKHKSSARNIIPKYSKDSKIVNQEGGKTLAEFPNSIELGKLGPGKDPNNKEHSTTSLVVFFLLVAMRTGVYL

SEQ ID NO: 17 - protein, 664 aa, synthetic construct
  REGION 1..664: note = Description of sequence: circular nucleic acid product
MLTSILIKGRTPVLASRNLLAALSNCNHITWAVSRRLYNDGNRDQRNFGRNQRNNNSNRYRNSRFNSRPRTRSREDDDEVHFDKTTFSKLIHVPKEDNSKEVTLDSLLEEGVLDKEIHKAITRMEFPGLTPVQQKTIKPILSSEDHDVIARAKTGTGKTFAFLIPIFQHLINTKFDSQYMVKAVIVAPTRDLALQIEAEVKKIHDMNYGLKKYACVSLVGGTDFRAAMNKMNKLRPNIVIATPGRLIDVLEKYSNKFFRFVDYKVLDEADRLLEIGFRDDLETISGILNEKNSKSADNIKTLLFSATLDDKVQKLANNIMNKKECLFLDTVDKNEPEAHERIDQSVVISEKFANSIFAAVEHIKKQIKERDSNYKAIIFAPTVKFTSFLCSILKNEFKKDLPILEFHGKITQNKRTSLVKRFKKDESGILVCTDVGARGMDFPNVHEVLQIGVPSELANYIHRIGRTARSGKEGSSVLFICKDELPFVRELEDAKNIVIAKQEKYEPSEEIKSEVLEAVTEEPEDISDIVISLISSYRSCIKEYRFSERRILPEIASTYGVLLNDPQLKIPVSRRFLDKLGLSRSPIGKAMFEIRDYSSRDGNNKSYDYDDDSEISFRGNKNYNNRSQNRDYDDEPFRRSNNNRRSFSRSNDKNNYSSRNSNIY

SEQ ID NO: 18 - DNA, 23 bp, synthetic construct
  misc_feature 1..23: note = Description of sequence: circular nucleic acid component
  source 1..23: mol_type = other DNA
gcgaacgcattctggcgtagacc

SEQ ID NO: 19 - DNA, 179 bp, synthetic construct
  misc_feature 1..179: note = Description of sequence: circular nucleic acid component
  source 1..179: mol_type = other DNA
ggcggctgtgcgaacgcattctggcgtagaccggtaaaaacaaaaaacataggtcaagatattgttgagtttatttgaggaggtttctctgtaaataatgcaactttaaacagattctttgcattgttttttgtttttatccgttgaccttaaacggtcgtgtgggttcaagtccctcc

SEQ ID NO: 20 - DNA, 179 bp, synthetic construct
  misc_feature 1..179: note = Description of sequence: circular nucleic acid component
  source 1..179: mol_type = other DNA
ccgccgacacgcttgcgtaagaccgcatctggccatttttgttttttgtatccagttctataacaactcaaataaactcctccaaagagacatttattacgttgaaatttgtctaagaaacgtaacaaaaaacaaaaataggcaactggaatttgccagcacacccaagttcagggagg

SEQ ID NO: 21 - DNA, 89 bp, synthetic construct
  misc_feature 1..89: note = Description of sequence: circular nucleic acid component
  source 1..89: mol_type = other DNA
ttctggcgtagaccggtaaaaacaaaaaacttgagtttatttgaggaggtttctctgtaaataatgcaacgttttttgtttttggatcc

SEQ ID NO: 22 - DNA, 89 bp, synthetic construct
  misc_feature 1..89: note = Description of sequence: circular nucleic acid component
  source 1..89: mol_type = other DNA
aagaccgcatctggccatttttgttttttgaactcaaataaactcctccaaagagacatttattacgttgcaaaaaacaaaaacctagg

SEQ ID NO: 23 - DNA, 74 bp, synthetic construct
  misc_feature 1..74: note = Description of sequence: circular nucleic acid component
  source 1..74: mol_type = other DNA
cctaaccgtcgattgtccactggtcccataaccatatcagaccagtggacaatcgacggggtagtaaactacta

SEQ ID NO: 24 - DNA, 74 bp, synthetic construct
  misc_feature 1..74: note = Description of sequence: circular nucleic acid component
  source 1..74: mol_type = other DNA
ggattggcagctaacaggtgaccagggtattggtatagtctggtcacctgttagctgccccatcatttgatgat

SEQ ID NO: 25 - DNA, 75 bp, synthetic construct
  misc_feature 1..75: note = Description of sequence: circular nucleic acid component
  source 1..75: mol_type = other DNA
  variation 1: note = n is a t g or c
nctaaccgttcgattgtccactggtcccataaccatatcagaccagtggacaatcgacggggtagtaaactacta

SEQ ID NO: 26 - DNA, 72 bp, synthetic construct
  misc_feature 1..72: note = Description of sequence: circular nucleic acid component
  source 1..72: mol_type = other DNA
aaccgttcgattgtccactggtcccataaccatatcagaccagtggacaatcgacggggtagtaaactacta

SEQ ID NO: 27 - DNA, 177 bp, synthetic construct
  misc_feature 1..177: note = Description of sequence: circular nucleic acid component
  source 1..177: mol_type = other DNA
acggggaaaatcagcaggggtgagaagtttacatacttcagaagcctctcagagactacatgcaggagatcttatgtcgacataagatcatgatatagtccgatcaataaagaaatttattgcgtatagtaagaggatttaatatttatattaaatctgtaactatcaacataaatg

SEQ ID NO: 28 - DNA, 134 bp, synthetic construct
  misc_feature 1..134: note = Description of sequence: circular nucleic acid component
  source 1..134: mol_type = other DNA
atacacagatgaaccacaatgcggtgacgtattgttaaaaatcctgcttaatgctggaaaatccccaatcttaggatttgcatacgacttattctttataatagtattattaataggcgtgaaaattgcaatga

SEQ ID NO: 29 - DNA, 135 bp, synthetic construct
  misc_feature 1..135: note = Description of sequence: circular nucleic acid component
  source 1..135: mol_type = other DNA
atacacagatgaaccacaatgcggtgacgtattgttaaaaatcctgcttaatgctggaaaatccccaatcttaggatttgcatacgacttattctttataatagtattattaataggcgtgaaaattgcaatgag

SEQ ID NO: 30 - DNA, 197 bp, synthetic construct
  misc_feature 1..197: note = Description of sequence: circular nucleic acid component
  source 1..197: mol_type = other DNA
ggcgtgaaaattgcaatgacacggggaaaatcagcaggggtgagaagtttacatacttcagaagcctctcagagactacatgcaggagatcttatgtcgacataagatcatgatatagtccgatcaataaagaaatttattgcgtatagtaagaggatttaatatttatattaaatctgtaactatcaacataaatg

SEQ ID NO: 31 - DNA, 116 bp, synthetic construct
  misc_feature 1..116: note = Description of sequence: circular nucleic acid component
  source 1..116: mol_type = other DNA
atacacagatgaaccacaatgcggtgacgtattgttaaaaatcctgcttaatgctggaaaatccccaatcttaggatttgcatacgacttattctttataatagtattattaatag

SEQ ID NO: 32 - DNA, 255 bp, synthetic construct
  misc_feature 1..255: note = Description of sequence: circular nucleic acid component
  source 1..255: mol_type = other DNA
tcctgcttaatgctggaaaatccccaatcttaggatttgcatacgacttattctttataatagtattattaataggcgtgaaaattgcaatgacacggggaaaatcagcaggggtgagaagtttacatacttcagaagcctctcagagactacatgcaggagatcttatgtcgacataagatcatgatatagtccgatcaataaagaaatttattgcgtatagtaagaggatttaatatttatattaaatctgta

SEQ ID NO: 33 - DNA, 42 bp, synthetic construct
  misc_feature 1..42: note = Description of sequence: circular nucleic acid component
  source 1..42: mol_type = other DNA
atacacagatgaaccacaatgcggtgacgtattgttaaaaag

SEQ ID NO: 34 - DNA, 120 bp, synthetic construct
  misc_feature 1..120: note = Description of sequence: circular nucleic acid component
  source 1..120: mol_type = other DNA
acatgcaggagatcttatgtcgacataagatcatgatatagtccgatcaataaagaaatttattgcgtatagtaagaggatttaatatttatattaaatctgtaactatcaacataaatg

SEQ ID NO: 35 - DNA, 192 bp, synthetic construct
  misc_feature 1..192: note = Description of sequence: circular nucleic acid component
  source 1..192: mol_type = other DNA
atacacagatgaaccacaatgcggtgacgtattgttaaaaatcctgcttaatgctggaaaatccccaatcttaggatttgcatacgacttattctttataatagtattattaataggcgtgaaaattgcaatgacacggggaaaatcagcaggggtgagaagtttacatacttcagaagcctctcagagact

SEQ ID NO: 36 - DNA, 741 bp, synthetic construct
  misc_feature 1..741: note = Description of sequence: circular nucleic acid component
  source 1..741: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 37 - DNA, 13 bp, synthetic construct
  misc_feature 1..13: note = Description of sequence: circular nucleic acid component
  source 1..13: mol_type = other DNA
gttttttgttttt

SEQ ID NO: 38 - DNA, 39 bp, synthetic construct
  misc_feature 1..39: note = Description of sequence: circular nucleic acid component
  source 1..39: mol_type = other DNA
ctctgtaaataatgcaactttaaacagattctttgcatt

SEQ ID NO: 39 - DNA, 18 bp, synthetic construct
  misc_feature 1..18: note = Description of sequence: circular nucleic acid component
  source 1..18: mol_type = other DNA
ctctgtaaataatgcaac

SEQ ID NO: 40 - DNA, 255 bp, synthetic construct
  misc_feature 1..255: note = Description of sequence: circular nucleic acid component
  source 1..255: mol_type = other DNA
gtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggat

SEQ ID NO: 41 - DNA, 13 bp, synthetic construct
  misc_feature 1..13: note = Description of sequence: circular nucleic acid component
  source 1..13: mol_type = other DNA
aaaaacaaaaaac

SEQ ID NO: 42 - DNA, 38 bp, synthetic construct
  misc_feature 1..38: note = Description of sequence: circular nucleic acid component
  source 1..38: mol_type = other DNA
ataggtcaagatattgttgagtttatttgaggaggttt

SEQ ID NO: 43 - DNA, 22 bp, synthetic construct
  misc_feature 1..22: note = Description of sequence: circular nucleic acid component
  source 1..22: mol_type = other DNA
ttgagtttatttgaggaggttt

SEQ ID NO: 44 - DNA, 68 bp, synthetic construct
  misc_feature 1..68: note = Description of sequence: circular nucleic acid component
  source 1..68: mol_type = other DNA
atccgttgaccttaaacggtcgtgtgggttcaagtccctccacccccacgccggaaacgcaatagccg

SEQ ID NO: 45 - RNA, 2544 nt, synthetic construct
  misc_feature 1..2544: note = Description of sequence: circular nucleic acid component
  source 1..2544: mol_type = other RNA
gcgaatatgaatttgtgattgcaacccacgtcgatcgtccataacgtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcggttatgtgtcgatagaggaaagtgtctgaaacctctagtacaaagaaaggaaagttatggttgtggacttttctgttatcaccacatttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgtatcaaaatttatattccaataaaggagcttccacaaaaggaatattagatgatacagcggatggctttagtgaagaaaaaataaaaaagattattcaatctttaaaagacggaacttactatcctcaacctgtacgaagaatgtatattgcaaaaaagaattctaaaaagatgagacctttaggaattccaactttcacagataaattgatccaagaagctgtgagaataattcttgaatctatctatgaaccggtattcgaagatgtgtctcacggttttagacctcaacgaagctgtcacacagctttgaaaacaatcaaaagagagtttggcggcgcaagatggtttgtggagggagatataaaaggctgcttcgataatatagaccacgttacactcattggactcatcaatcttaaaatcaaagatatgaaaatgagccaattgatttataaatttctaaaagcaggttatctggaaaactggcagtatcacaaaacttacagcggaacacctcaaggtggaattctatctcctcttttggccaacatctatcttcatgaattggataagtttgttttacaactcaaaatgaagtttgaccgagaaagtccagaaagaataacacctgaatatcgggaacttcacaatgagataaaaagaatttctcaccgtctcaagaagttggagggtgaagaaaaagctaaagttcttttagaatatcaagaaaaacgtaaaagattacccacactcccctgtacctcacagacaaataaagtattgaaatacgtccggtatgcggacgacttcattatctctgttaaaggaagcaaagaggactgtcaatggataaaagaacaattaaaactttttattcataacaagctaaaaatggaattgagtgaagaaaaaacactcatcacacatagcagtcaacccgctcgttttctgggatatgatatacgagtaaggagaagtggaacgataaaacgatctggtaaagtcaaaaagagaacactcaatgggagtgtagaactccttattcctcttcaagacaaaattcgtcaatttatttttgacaagaaaatagctatccaaaagaaagatagctcatggtttccagttcacaggaaatatcttattcgttcaacagacttagaaatcatcacaatttataattctgaattaagagggatttgtaattactacggtctagcaagtaattttaaccagctcaattattttgcttatcttatggaatacagctgtctaaaaacgatagcctccaaacataagggaacactttcaaaaaccatttccatgtttaaagatggaagtggttcgtggggcatcccgtatgagataaagcaaggtaagcagcgccgttattttgcaaattttagtgaatgtaaatccccttatcaatttacggatgagataagtcaagctcctgtattgtatggctatgcccggaatactcttgaaaacaggttaaaagctaaatgttgtgaattatgtggaacatctgatgaaaatacttcctatgaaattcaccatgtcaataaggtcaaaaatcttaaaggcaaagaaaaatgggaaatggcaatgatagcgaaacaacgtaaaactcttgttgtatgctttcattgtcatcgtcacgtgattcataaacacaagtgaatttttacgaacgaacaataacagagccgtatactccgagaggggtacgtacggttcccgaagagggtggtgcaaaccagtcacagtaatgtgaacaaggcggtacctccctacttcaccatatca

SEQ ID NO: 46 - RNA, 3901 nt, synthetic construct
  misc_feature 1..3901: note = Description of sequence: circular nucleic acid component
  source 1..3901: mol_type = other RNA
gggtacgttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgtatcaaaatttatattccaataaaggagcttccacaaaaggaatattagatgatacagcggatggctttagtgaagaaaaaataaaaaagattattcaatctttaaaagacggaacttactatcctcaacctgtacgaagaatgtatattgcaaaaaagaattctaaaaagatgagacctttaggaattccaactttcacagataaattgatccaagaagctgtgagaataattcttgaatctatctatgaaccggtattcgaagatgtgtctcacggttttagacctcaacgaagctgtcacacagctttgaaaacaatcaaaagagagtttggcggcgcaagatggtttgtggagggagatataaaaggctgcttcgataatatagaccacgttacactcattggactcatcaatcttaaaatcaaagatatgaaaatgagccaattgatttataaatttctaaaagcaggttatctggaaaactggcagtatcacaaaacttacagcggaacacctcaaggtggaattctatctcctcttttggccaacatctatcttcatgaattggataagtttgttttacaactcaaaatgaagtttgaccgagaaagtccagaaagaataacacctgaatatcgggaacttcacaatgagataaaaagaatttctcaccgtctcaagaagttggagggtgaagaaaaagctaaagttcttttagaatatcaagaaaaacgtaaaagattacccacactcccctgtacctcacagacaaataaagtattgaaatacgtccggtatgcggacgacttcattatctctgttaaaggaagcaaagaggactgtcaatggataaaagaacaattaaaactttttattcataacaagctaaaaatggaattgagtgaagaaaaaacactcatcacacatagcagtcaacccgctcgttttctgggatatgatatacgagtaaggagaagtggaacgataaaacgatctggtaaagtcaaaaagagaacactcaatgggagtgtagaactccttattcctcttcaagacaaaattcgtcaatttatttttgacaagaaaatagctatccaaaagaaagatagctcatggtttccagttcacaggaaatatcttattcgttcaacagacttagaaatcatcacaatttataattctgaattaagagggatttgtaattactacggtctagcaagtaattttaaccagctcaattattttgcttatcttatggaatacagctgtctaaaaacgatagcctccaaacataagggaacactttcaaaaaccatttccatgtttaaagatggaagtggttcgtggggcatcccgtatgagataaagcaaggtaagcagcgccgttattttgcaaattttagtgaatgtaaatccccttatcaatttacggatgagataagtcaagctcctgtattgtatggctatgcccggaatactcttgaaaacaggttaaaagctaaatgttgtgaattatgtggaacatctgatgaaaatacttcctatgaaattcaccatgtcaataaggtcaaaaatcttaaaggcaaagaaaaatgggaaatggcaatgatagcgaaacaacgtaaaactcttgttgtatgctttcattgtcatcgtcacgtgattcataaacacaagtgaatttttacgaacgaacaataacagagccgtatactccgagaggggtacgtacggttcccgaagagggtggtgcaaaccagtcacagtaatgtgaacaaggcggtacctccctacttcaccatatcagaccagtggacaatcgacggggtagtaaactactaactacaacctgctgaagcaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggttttcaccttggaagatttcgtcggggattggcgtcaaaccgcaggttacaacctcgatcaggttttggaacagggcggggtaagttctctcttccaaaatcttggagtctccgtcacgcctatccagagaattgttttgtcaggcgaaaatggattgaaaattgatattcatgtcattataccttatgagggtctgagcggggaccaaatgggacagattgaaaagattttcaaagtcgtctacccagtcgacgaccatcatttcaaggtgattttgcattatggaacactggtcattgatggagtgacccctaacatgatagactacttcggccgcccctatgaggggatagcggtgttcgacggtaagaaaataacagtcaccggcactttgtggaatggcaataaaatcatcgatgaaagattgatcaatcctgatggctcactgttgtttagggttacaataaacggggtaacgggatggcggctctgcgaacggattcttgccgactacaaagaccatgacggtgattataaagatcatgatatcgattacaaggatgacgatgacaagtgaggatcctaaccgtcgattgtccactggtcccataacgtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcggttatgtgtcgatagaggaaagtgtctgaaacctctagtacaaagaaaggaaagttatggttgtggacttttctgttatcaccacatttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactc

SEQ ID NO: 47 - RNA, 2088 nt, synthetic construct
  misc_feature 1..2088: note = Description of sequence: circular nucleic acid component
  source 1..2088: mol_type = other RNA
acaataacagagccgtatactccgagaggggtacgtacggttcccgaagagggtggtgcaaaccagtcacagtaatgtgaacaaggcggtacctccctacttcaccatatcagaccagtggacaatcgacggggtagtaaactactaactacaacctgctgaagcaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggttttcaccttggaagatttcgtcggggattggcgtcaaaccgcaggttacaacctcgatcaggttttggaacagggcggggtaagttctctcttccaaaatcttggagtctccgtcacgcctatccagagaattgttttgtcaggcgaaaatggattgaaaattgatattcatgtcattataccttatgagggtctgagcggggaccaaatgggacagattgaaaagattttcaaagtcgtctacccagtcgacgaccatcatttcaaggtgattttgcattatggaacactggtcattgatggagtgacccctaacatgatagactacttcggccgcccctatgaggggatagcggtgttcgacggtaagaaaataacagtcaccggcactttgtggaatggcaataaaatcatcgatgaaagattgatcaatcctgatggctcactgttgtttagggttacaataaacggggtaacgggatggcggctctgcgaacggattcttgccgactacaaagaccatgacggtgattataaagatcatgatatcgattacaaggatgacgatgacaagtgaggatcctaaccgtcgattgtccactggtcccataacgtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcggttatgtgtcgatagaggaaagtgtctgaaacctctagtacaaagaaaggaaagttatggttgtggacttttctgttatcaccacatttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactcgtagtagtctgagaagggtaacgccctttacatggcaaaggggtacagttattgt

SEQ ID NO: 48 - RNA, 1960 nt, synthetic construct
  misc_feature 1..1960: note = Description of sequence: circular nucleic acid component
  source 1..1960: mol_type = other RNA
acaataacagagccgtatactccgagaggggtacgtacggttcccgaagagggtggtgcaaaccagtcacagtaatgtgaacaaggcggtacctccctacttcaccatatcagaccagtggacaatcgacggggtagtaaactactaactacaacctgctgaagcaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggttttcaccttggaagatttcgtcggggattggcgtcaaaccgcaggttacaacctcgatcaggttttggaacagggcggggtaagttctctcttccaaaatcttggagtctccgtcacgcctatccagagaattgttttgtcaggcgaaaatggattgaaaattgatattcatgtcattataccttatgagggtctgagcggggaccaaatgggacagattgaaaagattttcaaagtcgtctacccagtcgacgaccatcatttcaaggtgattttgcattatggaacactggtcattgatggagtgacccctaacatgatagactacttcggccgcccctatgaggggatagcggtgttcgacggtaagaaaataacagtcaccggcactttgtggaatggcaataaaatcatcgatgaaagattgatcaatcctgatggctcactgttgtttagggttacaataaacggggtaacgggatggcggctctgcgaacggattcttgccgactacaaagaccatgacggtgattataaagatcatgatatcgattacaaggatgacgatgacaagtgaggatcctaaccgtcgattgtccactggtcccataacgtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcggttatgtgtcgatagaggaaagtgtctgaaacctctagtacaaagaaaggaaagttatggttgtggacttttctgttatcaccacatttgtacaatctgtaggagaacctatgggaacgaaacgaaagcgatgccgagaatctgaatttaccaagacttaacactaactgggacggaggttattgt

SEQ ID NO: 49 - RNA, 826 nt, synthetic construct
  misc_feature 1..826: note = Description of sequence: circular nucleic acid component
  source 1..826: mol_type = other RNA
gaaataggtcgtcaaaccatattagaatttacaggtggcgaatatgaatttgtgattgcaacccacgtcgatcgtgaacacatccataacgtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcggttatgtgtcgatagaggaaagtgtctgaaacctctagtacaaagaaaggaaagttatggttgtggacttttctgttatcaccacatttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactcgtagtagtctgagaagggtaacgccctttacatggcaaaggggtacagttattgtacaataacagagccgtatactccgagaggggtacgtacggttcccgaagagggtggtgcaaaccagtcacagtaatgtgaacaaggcggtacctccctacttcaccatatcatttttaattctacgaatctttatactggcaaacaatttgactggaaagtcattcctaaagagaaaacaaaaagcggcaaagcttatgatgt

SEQ ID NO: 50 - RNA, 2118 nt, synthetic construct
  misc_feature 1..2118: note = Description of sequence: circular nucleic acid component
  source 1..2118: mol_type = other RNA
acaataacagagccgtatactccgagaggggtacgtacggttcccgaagagggtggtgcaaaccagtcacagtaatgtgaacaaggcggtacctccctacttcaccatatcatttttaattctacgagaccagtggacaatcgacggggtagtaaactactaactacaacctgctgaagcaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggttttcaccttggaagatttcgtcggggattggcgtcaaaccgcaggttacaacctcgatcaggttttggaacagggcggggtaagttctctcttccaaaatcttggagtctccgtcacgcctatccagagaattgttttgtcaggcgaaaatggattgaaaattgatattcatgtcattataccttatgagggtctgagcggggaccaaatgggacagattgaaaagattttcaaagtcgtctacccagtcgacgaccatcatttcaaggtgattttgcattatggaacactggtcattgatggagtgacccctaacatgatagactacttcggccgcccctatgaggggatagcggtgttcgacggtaagaaaataacagtcaccggcactttgtggaatggcaataaaatcatcgatgaaagattgatcaatcctgatggctcactgttgtttagggttacaataaacggggtaacgggatggcggctctgcgaacggattcttgccgactacaaagaccatgacggtgattataaagatcatgatatcgattacaaggatgacgatgacaagtgaggatcctaaccgtcgattgtccactggtccgatcgtgaacacatccataacgtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcggttatgtgtcgatagaggaaagtgtctgaaacctctagtacaaagaaaggaaagttatggttgtggacttttctgttatcaccacatttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactcgtagtagtctgagaagggtaacgccctttacatggcaaaggggtacagttattgt

SEQ ID NO: 51 - RNA, 2103 nt, synthetic construct
  misc_feature 1..2103: note = Description of sequence: circular nucleic acid component
  source 1..2103: mol_type = other RNA
acaataacagagccgtatactccgagaggggtacgtacggttcccgaagagggtggtgcaaaccagtcacagtaatgtgaacaaggcggtacctccctacttcaccatatcagaccagtggacaatcgacggggtagtaaactactaactacaacctgctgaagcaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggttttcaccttggaagatttcgtcggggattggcgtcaaaccgcaggttacaacctcgatcaggttttggaacagggcggggtaagttctctcttccaaaatcttggagtctccgtcacgcctatccagagaattgttttgtcaggcgaaaatggattgaaaattgatattcatgtcattataccttatgagggtctgagcggggaccaaatgggacagattgaaaagattttcaaagtcgtctacccagtcgacgaccatcatttcaaggtgattttgcattatggaacactggtcattgatggagtgacccctaacatgatagactacttcggccgcccctatgaggggatagcggtgttcgacggtaagaaaataacagtcaccggcactttgtggaatggcaataaaatcatcgatgaaagattgatcaatcctgatggctcactgttgtttagggttacaataaacggggtaacgggatggcggctctgcgaacggattcttgccgactacaaagaccatgacggtgattataaagatcatgatatcgattacaaggatgacgatgacaagtgaggatcctaaccgtcgattgtccactggtccgatcgtgaacacatccataacgtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcggttatgtgtcgatagaggaaagtgtctgaaacctctagtacaaagaaaggaaagttatggttgtggacttttctgttatcaccacatttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactcgtagtagtctgagaagggtaacgccctttacatggcaaaggggtacagttattgt